(19) United States
(12) Patent Application Publication
(10) Pub. No.: US 2019/0321311 A1
(43) Pub. Date: Oct. 24, 2019

(54) Cancer Treatment by Simultaneous Targeting Energy Metabolism and Intracellular pH

(71) Applicant: Deutsches Krebsforschungszentrum, Heidelberg (DE)

(72) Inventors: Andrey Glinka, Schriesheim (DE); Svitlana Melnik, Schriesheim (DE); Dmytro Dvornikov, Heidelberg (DE); Christof Niehrs, Mainz (DE)

(21) Appl. No.: 16/310,379

(22) PCT Filed: Jun. 14, 2017
(86) PCT No.: PCT/EP2017/064606

§ 371 Date: Dec. 14, 2018

(30) Foreign Application Priority Data
Jun. 15, 2016 (EP) 16174558.3

(51) Int. Cl. A61K 31/155; A61K 31/35; A61K 31/353; A61K 31/472; A61K 31/65; A61P 35/00

(52) U.S. Cl. CPC A61K 31/155; A61K 31/35; A61P 35/00; A61K 31/472; A61K 31/65; A61K 31/353

(57) ABSTRACT

The present invention relates to an inhibitor of mitochondrial respiration for use in treatment of cancer with a proton ionophore and relates to a proton ionophore for use in treatment of cancer with an inhibitor of mitochondrial respiration. The present invention further relates to a combined preparation for simultaneous, separate or sequential use comprising (i) an inhibitor of mitochondrial respiration and (ii) a proton ionophore, to said combined preparation for use in treatment of cancer, and to kits and methods related thereto.

The present invention relates to an inhibitor of mitochondrial respiration for use in treatment of cancer with a proton ionophore and relates to a proton ionophore for use in treatment of cancer with an inhibitor of mitochondrial respiration. The present invention further relates to a combined preparation for simultaneous, separate or sequential use comprising (i) an inhibitor of mitochondrial respiration and (ii) a proton ionophore, to said combined preparation for use in treatment of cancer, and to kits and methods related thereto.

Cancer constitutes the fourth leading cause of death in Western countries. As the average age in the Western population steadily rises, so do cancer-related deaths indicating that cancer will be one of the most common causes of death in the 21st century. The aggressive cancer cell phenotype is the result of a variety of genetic and epigenetic alterations leading to deregulation of intracellular signaling pathways. Cancer cells commonly fail to undergo so-called “programmed cell death” or “apoptosis”, a signaling process that plays a key role in preventing cell tissues from abnormal growth.

Three modes of cancer therapy are generally available. Curative surgery attempts to remove the tumor completely. This is only possible as long as there are no metastases. Sometimes surgery may be an option for the treatment of metastases if there are only few and they are easily accessible. Radiotherapy uses ionizing radiation, typically γ-radiation, to destroy the tumor. Radiation therapy is based on the principle that tumor cells with their high metabolic rates are especially susceptible to radiation induced cell damage. The anti-tumor effect of radiation therapy has to be weighted against the damage to the surrounding healthy tissue. Thus, possible tissue damage can rule out this option in some cases due to the damage to healthy tissues to be feared. Furthermore, radiation therapy is limited to cases where the primary tumor has not yet spread or where only few metastases are present.

The most commonly used—and in many instances the only available—systemic treatment for cancer is chemotherapy. For patients suffering from leukemia or from metastases of solid tumors, thus, chemotherapy is the only treatment option. Chemotherapeutic agents are cytotoxic for all rapidly dividing cells. As cancer cells usually divide more rapidly than other cells in the body, they are preferably killed by these agents. Common groups of chemotherapeutic agents are substances that inhibit cell division by interfering with the formation of the mitotic spindle or agents which damage the DNA, e.g. by alkylating the bases. Because all rapidly dividing cells are targeted by chemotherapeutic agents, their side effects are usually severe. Depending on the substance used, they include organ toxicity (e.g. heart or kidney), immunosuppression, neurotoxicity and anemia. Some groups of chemotherapeutic agents, e.g. alkylating agents, even have the potential to cause cancer. Due to these side effects, dosages have sometimes to be reduced or chemotherapy has to be discontinued completely. Furthermore, the side effects of chemotherapy often prohibit the treatment of patients in a bad general condition. Adding to all these problems is the often limited efficacy of chemotherapy. In some cases chemotherapy fails from the very beginning. In other cases, tumor cells become resistant during the course of treatment. To combat the emergence of resistant tumor cells and to limit the side effects of chemotherapy, combinations of different compounds with different modes of action are used. Nevertheless, the success of chemotherapy has been limited, especially in the treatment of solid tumors.

Recently, drugs have become available whose mode of action is not based on toxicity against rapidly dividing cells. These compounds show a higher specificity for cancer cells and thus less side effects than conventional chemotherapeutic agents. Imatinib is used for the specific treatment of chronic myelogenous leukemia. This compound specifically inhibits an abnormal tyrosine kinase which is the product of a fusion gene of bcr and abl. Because this kinase does not occur in non-malignant cells, treatment with Imatinib has only mild side effects. However, Imatinib is not used for the treatment of hematological cancers other than myelogenous leukemia. Rituximab is a monoclonal antibody directed against the cluster of differentiation 20 (CD20), which is widely expressed on B-cells. It is used for the treatment of B cell lymphomas in combination with conventional chemotherapy.

Attempts for targeting energy metabolism of cancer cells have been made, e.g. by using the non-metabolizable glucose analog 2-deoxyglucose; clinical use of this compound, however, is hampered by its side effects (Singh et al., Strahlenther Onkol. 2005; 181(8):507-14; Marsh et al., Nutr Metab (Lond). 2008; 5:33).

In noncancerous cells, pH outside of the plasma membrane is about 7.4 and intracellular pH is 7.2. It is known that cancer cells create a reversed pH gradient. The extracellular pH in a cancer cell is 6.5, however, the intracellular pH is maintained in a range of 7.2-7.4 (M. Damaghi et al. (2013), Frontiers in Physiology v.4, 370). In case of intracellular acidification reaching a pH in the range of 7.0-6.8, irreversible processes are induced in the cell leading to its death (D Lagadic-Gossmann et al. (2004), Cell Death and Differentiation 11, 953-961).

Metformin is the first-line oral drug used for treatment of millions of diabetes Type II patients worldwide. Epidemiological studies established a link between intake of Metformin and a lower risk of cancer incidence for many types of malignancies (Wu et al. (2015), Scientific reports 5:10147). Despite of numerous investigations, anticancer mechanism of Metformin remains elusive. Previous studies had suggested that AMPK activation mediates anticancer action of Metformin (Li et al. (2015), Oncotarget 6:7365; Song et al. (2012), Scientific reports 2:362). This notion, however, is contradicted by an increasing number of reports showing the AMPK-independent anticancer (Vincent et al. (2015), Oncogene 34(28):3627-39) and antidiabetic action of Metformin (Foretz et al. (2014), Cell metabolism 20:953). As an alternative model, it was proposed that metformin inhibits mitochondrial complex I of cancer cells (Liu et al. (2012), Oncology reports 28:1406). An important consequence of treating cancer cells with Metformin is depletion of all nucleotide triphosphates (NTPs), including the main cellular energy equivalent, ATP (Janzer A. et al., 2014, PNAS 111(29):10574-9).

In view of the above, there is a need in the art for an improved cancer therapy, in particular cancer therapy targeting energy metabolism of a cancer cell, which preferably avoids or largely avoids the drawback of the prior art.

The technical problem underlying the present invention can be regarded as the provision of means and methods for complying with the aforementioned needs. The said technical problem is solved by the embodiments characterized in the claims and herein below.

Accordingly, the present invention relates to an inhibitor of mitochondrial respiration for use in treatment of cancer with a proton ionophore.

As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment of the invention” or similar expressions are intended to be optional features, without any restriction regarding further embodiments of the invention, without any restrictions regarding the scope of the invention and without any restriction regarding the possibility of combining the features introduced in such way with other optional or non-optional features of the invention. Moreover, if not noted otherwise, the term “about” relates to the indicated value ±20%. Also, if an effect is referred to as being significant, this term relates to statistical significance.

The term “mitochondrial respiration”, as used herein, relates to the biochemical reactions regenerating energy equivalents, preferably nucleotide triphosphates (NTPs), more preferably adenosine-triphosphate (ATP), in a mitochondrion of an animal cell. Preferably, mitochondrial respiration is mitochondrial oxidative phosphorylation, i.e. oxidation of redox equivalents to H₂O catalyzed by membrane-bound enzymes complexes of a mitochondrion, generating a proton gradient over the inner mitochondrial membrane usable by ATP synthase for regenerating ATP.

In accordance with the above, the term “inhibitor of mitochondrial respiration”, as used herein, relates to a chemical compound inhibiting mitochondrial respiration as specified herein above. Preferably, the inhibitor of mitochondrial respiration is an inhibitor of mitochondrial complex I (NADH-coenzyme Q oxidoreductase), of mitochondrial complex III (Q-cytochrome c oxidoreductase), of mitochondrial complex V (ATP synthase). Respective inhibitors of mitochondrial complexes are known in the art, e.g. rotenone as inhibitor of complex I (cf., e.g. Degli Esposti (1998), Biochimica et Biophysica Acta 1364:222), antimycin as inhibitor of complex III, and oligomycins as inhibitors of ATP synthase. Still more preferably, the inhibitor of mitochondrial respiration is an inhibitor of mitochondrial complex I; even more preferably, the inhibitor of mitochondrial respiration is selected from the list consisting of (i) Papaverine (CAS Number: 61-25-6), (ii) Rotenone (CAS Number: 83-79-4), (iii) Annonacin (CAS Number:111035-65-5), (iv) 1-methyl 4-phenyl 1,2,3,6 tetrahydropyridine (CAS Number 23007-85-4), (v) 3-nitropropionic acid (CAS Number: 504-88-1), (vi) Piericidin A (CAS Number 2738-64-9), (vii) Bullatacin A (CAS Number 123123-32-0), (viii) Rolliniastatin-1 ((2S)-4-[(2R,13R)-2,13-dihydroxy-13-[(5S)-5-[(2S)-5-[(1S)-1-hydroxyundecyl]oxolan-2-yl]oxolan-2-yl]tridecyl]-2-methyl-2H-furan-5-one), (ix) Phenoxan (CAS No. 134332-63-1), (x) Thiangazole (CAS No. 138667-71-7), (xi) Idebenone CAS No. 58186-27-9), (xii) Aureothin (CAS No 2825-00-5), (xiii) β-lapachone, (xiv) a derivative of any one of (i) to (xiii), (xv) a pharmaceutically acceptable salt of any one of (i) to (xiv), or (xvi) a prodrug of any of any one of (i) to (xiii). Most preferably, the inhibitor of mitochondrial respiration is selected from the list consisting of (i) Papaverine, (ii) Rotenone, (iii) Annonacin, (iv) a derivative of any one of (i) to (iii), (v) a pharmaceutically acceptable salt of any one of (i) to (iii), or (vi) a prodrug of any of any one of (i) to (iii). In a preferred embodiment, the inhibitor of mitochondrial respiration is selected from the list consisting of (i) Papaverine (CAS Number: 61-25-6), (ii) Rotenone (CAS Number: 83-79-4), (iii) Annonacin (CAS Number:111035-65-5) (iv) 1-methyl 4-phenyl 1,2,3,6 tetrahydropyridine (CAS Number 23007-85-4), (v) 3-nitropropionic acid (CAS Number: 504-88-1), (vi) Piericidin A (CAS Number 2738-64-9), (vii) Bullatacin A (CAS Number 123123-32-0), (viii) Rolliniastatin-1 ((2S)-4-[(2R,13R)-2,13-dihydroxy-13-[(5S)-5-[(2S)-5-[(1S)-1-hydroxyundecyl]oxolan-2-yl]oxolan-2-yl]tridecyl]-2-methyl-2H-furan-5-one), (ix) Phenoxan (CAS No. 134332-63-1), (x) Thiangazole (CAS No. 138667-71-7), (xi) Idebenone CAS No. 58186-27-9), (xii) Aureothin (CAS No 2825-00-5), (xiii) β-lapachone, (xiv) Phenformin (CAS Number:114-86-3), (xv) Metformin (CAS Number: 657-24-9), (xvi) Buformin (CAS Number:692-13-7), (xvii) NT1014, (xviii) Bay 87-2243 (CAS Number: 1227158-85-1), (xix) Gossypol (CAS Number: 303-45-7), (xx) a derivative of any one of (i) to (xix), (xxi) a pharmaceutically acceptable salt of any one of (i) to (xx), or (xxii) a prodrug of any of any one of (i) to (xix). Most preferably, the inhibitor of mitochondrial respiration is selected from the list consisting of (i) Papaverine, (ii) Rotenone, (iii) Annonacin, (iv) Phenformin, Metformin, (v) Bay 87-2243, (vi) Gossypol, (vii) a derivative of any one of (i) to (vi), (viii) a pharmaceutically acceptable salt of any one of (i) to (vi), or (ix) a prodrug of any of any one of (i) to (vi). NT1014 is known to the skilled person from Zhang et al. (Journal of Hematology & Oncology (2016) 9:91).

The term “ionophore”, is used herein is used in its conventional meaning known to the skilled person and, preferably, relates to a chemical compound transporting ions over a biological membrane, preferably at least over the plasma membrane and/or the inner mitochondrial membrane, of an animal cell. More preferably, said chemical compound reversibly binds to and transports ions over a biological membrane. Accordingly, the term “proton ionophore”, as used herein, is also used in its conventional meaning known to the skilled person and, preferably, relates to a chemical compound reversibly binding to and transporting protons over a biological membrane, preferably at least over the plasma membrane and/or the inner mitochondrial membrane, of an animal cell. According to the present invention, it is not required that the proton ionophore is a specific proton ionophore, i.e. it is not required that the proton ionophore exclusively binds and transports protons over a biological membrane. Thus, preferably, the proton ionophore is a compound further binding and transporting ions different from protons, preferably alkali metal ions, as well. More preferably, the proton ionophore is a K⁺/H⁺ ionophore, like e.g. nigericin; or is a Na⁺/H⁺ ionophore, like e.g. monensin. Preferably, the proton ionophore is (I) Nigericin, (II) Salinomycin, (III) Monensin, (IV) Metformin, (V) Phenformin, (VI) Buformin, (VII) ionomycin, (VIII) carbonylcyanide-p-trifluoromethoxyphenylhydrazone (FCCP), (IX) carbonyl cyanide m-chlorophenyl hydrazone (CCCP), (X) Alborixin (CAS: 57760-36-8), (XI) Desmethylalborixin (X-206, CAS 36505-48-3), (XII) Grisorixin (CAS: 31357-58-1), (XIII) a derivative of any one of (I) to (XII), (XIV) a prodrug of any of any one of (I) to (XII), or (XV) a pharmaceutically acceptable salt of any one of (I) to (XIII). In a preferred embodiment, the proton ionophore is (I) Nigericin, (II) Salinomycin, (III) Monensin, (IV) Maduramicin, (V) Lasalocid, (VI) Narasin, (VII) ionomycin, (VIII) carbonylcyanide-p-trifluoromethoxyphenylhydrazone (FCCP), (IX) carbonyl cyanide m-chlorophenyl hydrazone (CCCP), (X) Alborixin (CAS: 57760-36-8), (XI) Desmethylalborixin (X-206, CAS 36505-48-3), (XII) Grisorixin (CAS: 31357-58-1), (XIII) Semduramicin, (XIV) a derivative of any one of (I) to (XIII), (XV) a prodrug of any of any one of (I) to (XIII), or (XVI) a pharmaceutically acceptable salt of any one of (I) to (XIV).

The term “derivative”, as used herein, relates to a compound similar in structure to the compound it is derived from. Preferably, a derivative is a compound obtainable from a compound of interest by at most three, preferably at most two, more preferably by one derivatization steps known to the skilled person. More preferably, a derivative is a compound obtainable from a compound of interest by at most three, preferably at most two, more preferably by one derivatization step selected from (i) alkylation, preferably N- and/or O-alkylation, preferably methylation, ethylation, propylation, or isopropylation; (ii) esterification, preferably of —COOH and/or —OPO₃H₂ groups, preferably acetylation, propionylation, iso-propionylation, or succinylation; (iii) amidation, preferably acetamidation; (iv) reduction, preferably of C═C, hydroxyl, and/or carbonyl groups; (v); oxidation, preferably of hydroxyl, C—H, and/or C—C groups. More preferably, a derivative is an N-methyl or N-ethyl derivative, a carboxylic acid acetate or succinylate, or an N-acetyl derivative. Preferably, a prodrug is a derivative as specified above.

The term “treating” refers to ameliorating the diseases or disorders referred to herein or the symptoms accompanied therewith to a significant extent. Said treating as used herein also includes an entire restoration of the health with respect to the diseases or disorders referred to herein. It is to be understood that treating as used in accordance with the present invention may not be effective in all subjects to be treated. However, the term shall require that, preferably, a statistically significant portion of subjects suffering from a disease or disorder referred to herein can be successfully treated. Whether a portion is statistically significant can be determined without further ado by the person skilled in the art using various well known statistic evaluation tools, e.g., determination of confidence intervals, p-value determination, Student's t-test, Mann-Whitney test etc. Details are found in Dowdy and Wearden, Statistics for Research, John Wiley & Sons, New York 1983. Preferred confidence intervals are at least 90%, at least 95%, at least 97%, at least 98% or at least 99%. The p-values are, preferably, 0.1, 0.05, 0.01, 0.005, or 0.0001. Preferably, the probability envisaged by the present invention allows that the diagnosis will be correct for at least 60%, at least 70%, at least 80%, or at least 90% of the subjects of a given cohort or population.

Preferably, treating is inhibition of growth of a tumor and/or metastases; more preferably, treating is causing a tumor and/or metastases to shrink. Also preferably, treating is metastasis prevention, i.e., preferably, is preventing cancer cells from establishing metastasis in locations of the body non-identical to the location of the primary tumor. In a preferred embodiment, treating is inducing cancer cell and/or tumor necrosis. In a further preferred embodiment, treatment comprises preventing embryonic pathway signaling, preferably Wnt and/or TGFbeta signaling. In a further preferred embodiment, treatment comprises inducing differentiation of cancer cells, preferably comprises induction of loss of stem cell properties of cancer cells.

“Cancer” in the context of this invention refers to a disease of an animal, including man, characterized by uncontrolled growth by a group of body cells (“cancer cells”). This uncontrolled growth may be accompanied by intrusion into and destruction of surrounding tissue and possibly spread of cancer cells to other locations in the body (“metastasis”). Moreover, cancer may entail recurrence of cancer cells after an initial treatment apparently removing cancer cells from a subject (“relapse”). Preferably, cancer cells are cancer stem cells. Preferably, cancer is not pancreas cancer.

Preferably, the cancer is selected from the list consisting of acute lymphoblastic leukemia, acute myeloid leukemia, adrenocortical carcinoma, aids-related lymphoma, anal cancer, appendix cancer, astrocytoma, atypical teratoid, basal cell carcinoma, bile duct cancer, bladder cancer, brain stem glioma, breast cancer, burkitt lymphoma, carcinoid tumor, cerebellar astrocytoma, cervical cancer, chordoma, chronic lymphocytic leukemia, chronic myelogenous leukemia, colon cancer, colorectal cancer, craniopharyngioma, endometrial cancer, ependymoblastoma, ependymoma, esophageal cancer, extracranial germ cell tumor, extragonadal germ cell tumor, extrahepatic bile duct cancer, gallbladder cancer, gastric cancer, gastrointestinal stromal tumor, gestational trophoblastic tumor, hairy cell leukemia, head and neck cancer, hepatocellular cancer, hodgkin lymphoma, hypopharyngeal cancer, hypothalamic and visual pathway glioma, intraocular melanoma, kaposi sarcoma, laryngeal cancer, medulloblastoma, medulloepithelioma, melanoma, merkel cell carcinoma, mesothelioma, mouth cancer, multiple endocrine neoplasia syndrome, multiple myeloma, mycosis fungoides, nasal cavity and paranasal sinus cancer, nasopharyngeal cancer, neuroblastoma, non-hodgkin lymphoma, non-small cell lung cancer, oral cancer, oropharyngeal cancer, osteosarcoma, ovarian cancer, ovarian epithelial cancer, ovarian germ cell tumor, ovarian low malignant potential tumor, papillomatosis, paranasal sinus and nasal cavity cancer, parathyroid cancer, penile cancer, pharyngeal cancer, pheochromocytoma, pituitary tumor, pleuropulmonary blastoma, primary central nervous system lymphoma, prostate cancer, rectal cancer, renal cell cancer, retinoblastoma, rhabdomyosarcoma, salivary gland cancer, sézary syndrome, skin cancer, small cell lung cancer, small intestine cancer, soft tissue sarcoma, squamous cell carcinoma, squamous neck cancer, testicular cancer, throat cancer, thymic carcinoma, thymoma, thyroid cancer, urethral cancer, uterine sarcoma, vaginal cancer, vulvar cancer, waldenström macroglobulinemia, and wilms tumor. More preferably the cancer is a tumor-forming cancer, i.e. is a solid cancer. Still more preferably, the cancer is colorectal carcinoma, non-small cell lung cancer, breast cancer, skin cancer, prostate cancer, or lung cancer.

Preferably, the cancer is a cancer sensitive to combined treatment with an inhibitor of mitochondrial respiration and a proton ionophore. The term “cancer sensitive to combined treatment with an inhibitor of mitochondrial respiration and a proton ionophore”, as used herein, relates to a cancer, wherein the cells of said cancer show a significant decrease in viability upon treatment with an inhibitor of mitochondrial respiration and a proton ionophore, measured with a conventional viability assay (e.g. one of the assays reviewed in book “Assay Guidance Manual”, G. S. Sittampalam at al., Bethesda, version 2016), preferably an ATP measurement based viability assay. Most preferably, sensitivity is determined determining Sox4 expression as described herein in the Examples.

More preferably, the cancer is a Wnt signaling-dependent cancer. Preferably, the cancer sensitive to combined treatment with an inhibitor of mitochondrial respiration and a proton ionophore is a Wnt signaling-dependent cancer. The term “Wnt signaling-dependent cancer”, as used herein, relates to a cancer, wherein the cells generate in nuclei and/or cytoplasm abnormally high amount of b-catenin. Preferably, a Wnt signaling-dependent cancer is identified by biopsy analysis, more preferably according to Gomez-Millan et al. 2014, BMC Cancer 14:192.

In a preferred embodiment, the cancer is TGFbeta signaling-dependent cancer. The term “TGFbeta signaling-dependent cancer” is known to the skilled person. In a further preferred embodiment, the cancer comprises or consists of cancer cells having embryonic properties, preferably cancer stem cells. Embryonic markers of cancer cells are known to the skilled person.

Also more preferably, the cancer is a cancer wherein cancer cells show an unfolded protein response upon administration of an inhibitor of mitochondrial respiration and a proton ionophore. Preferably, the cancer sensitive to combined treatment with an inhibitor of mitochondrial respiration and a proton ionophore is a cancer wherein cancer cells show an unfolded protein response upon administration of an inhibitor of mitochondrial respiration and a proton ionophore. Determining whether cells, in particular cancer cells, show an unfolded protein response upon administration of an inhibitor of mitochondrial respiration and a proton ionophore, preferably comprises contacting said cells with an inhibitor of mitochondrial respiration and a proton ionophore and determining at least one marker of unfolded protein response. More preferably, determining whether cells, in particular cancer cells, show an unfolded protein response upon administration of an inhibitor of mitochondrial respiration and a proton ionophore is performed according to the methods as specified herein above and in the Examples. Preferably, the marker of unfolded protein response is a gene product, e.g., preferably, an mRNA or a polypeptide, of a gene encoding a CHOP, preferably CHOP10; DDIT3; Gadd153; and/or CEBPZ polypeptide, more preferably is a gene product of the human gene encoding the CHOP polypeptide, most preferably is the human CHOP mRNA as disclosed in Genbank AccNo: AAH03637.1 GI:13177718. Also preferably, the cancer is a cancer wherein cancer cells show decreased Sox4 expression as specified herein upon administration of an inhibitor of mitochondrial respiration and a proton ionophore.

Advantageously, it was found in the work underlying the present invention that combined treatment of cancer with an inhibitor of mitochondrial respiration and a proton ionophore has a synergistic effect resulting in improved killing of cancer cells. Moreover, it was found that by concomitant treatment with an inhibitor of mitochondrial respiration, the dose of a proton ionophore can be reduced while achieving the same killing effect on cancer cells, and vice versa. Killing of cells was found to occur soon after intracellular pH drop, CHOP protein/RNA induction and decrease of protein/RNA levels for cancer marker Sox4.

It was also found that the combined treatment of cancer cells with an inhibitor of mitochondrial respiration and a proton ionophore has strong inhibitory effect on Wnt signaling in cancer cells. Metformin and drug combinations according to the invention block Wnt signaling epistatically downstream of b-catenin. This provides an additional possibility for treatment of wnt signaling-dependent cancers in particular, preferably colorectal carcinomas and lung cancer, most preferably colon cancer. Further, it was surprisingly found in the work underlying the present invention that Metformin induces intracellular acidification in cancer cells, but not in non-cancer cells.

The definitions made above apply mutatis mutandis to the following. Additional definitions and explanations made further below also apply for all embodiments described in this specification mutatis mutandis.

The present invention further relates to a proton ionophore for use in treatment of cancer with an inhibitor of mitochondrial respiration.

The present invention also relates to a combined preparation for simultaneous, separate or sequential use comprising (i) an inhibitor of mitochondrial respiration and (ii) a proton ionophore; and to said combined preparation for use in treatment of cancer.

The term “combined preparation”, as referred to in this application, relates to a preparation comprising the pharmaceutically active compounds of the present invention in one preparation. Preferably, the combined preparation is comprised in a container, i.e. preferably, said container comprises all pharmaceutically active compounds of the present invention.

Preferably, said container comprises the pharmaceutically active compounds of the present invention as separate formulations, i.e. preferably, one formulation of the inhibitor of mitochondrial respiration and one formulation of the proton ionophore. As will be understood by the skilled person, the term “formulation” relates to a, preferably pharmaceutically acceptable, mixture of compounds, comprising or consisting of at least one pharmaceutically active compound of the present invention. Preferably, the combined preparation comprises a proton ionophore and inhibitor of mitochondrial respiration in a single solid pharmaceutical form, e.g. a tablet, wherein, more preferably, one compound of the present invention is comprised in an immediate or fast release formulation, and the second compound of the present invention is comprised in a slow or retarded release formulation; more preferably, the compounds of the present invention are comprised in two separate, preferably liquid, formulations; said separate liquid formulations, preferably are for injection, more preferably at different parts of the body of a subject.

Preferably, the combined preparation is for separate or for combined administration. “Separate administration”, as used herein, relates to an administration wherein at least two of the pharmaceutically active compounds of the present invention are administered via different routes and/or at different parts of the body of a subject. E.g. one compound may be administered by enteral administration (e.g. orally), whereas a second compound is administered by parenteral administration (e.g. intravenously). Preferably, the combined preparation for separate administration comprises at least two physically separated preparations for separate administration, wherein each preparation contains at least one pharmaceutically active compound; said alternative is preferred e.g. in cases where the pharmaceutically active compounds of the combined preparation have to be administered by different routes, e.g. parenterally and orally, due to their chemical or physiological properties. Conversely, “combined administration” relates to an administration wherein the pharmaceutically active compounds of the present invention are administered via the same route, e.g. orally or intravenously.

Also preferably, the combined preparation is for simultaneous or for sequential administration. “Simultaneous administration”, as used herein, relates to an administration wherein the pharmaceutically active compounds of the present invention are administered at the same time, i.e., preferably, administration of the pharmaceutically active compounds starts within a time interval of less than 15 minutes, more preferably, within a time interval of less than 5 minutes. Most preferably, administration of the pharmaceutically active compounds starts at the same time, e.g. by swallowing a tablet comprising the pharmaceutically active compounds, or by swallowing a tablet comprising one of the pharmaceutically active compounds and simultaneous injection of the second compound, or by applying an intravenous injection of a solution comprising one pharmaceutically active compound and injecting second compound in different part of the body. Conversely, “sequential administration, as used herein, relates to an administration causing plasma concentrations of the pharmaceutically active compounds in a subject enabling the synergistic effect of the present invention, but which, preferably, is not a simultaneous administration as specified herein above. Preferably, sequential administration is an administration wherein administration of the pharmaceutically active compounds, preferably all pharmaceutically active compounds, starts within a time interval of 1 or 2 days, more preferably within a time interval of 12 hours, still more preferably within a time interval of 4 hours, even more preferably within a time interval of one hour, most preferably within a time interval of 5 minutes.

Preferably, the combined preparation is a pharmaceutically compatible combined preparation. The terms “pharmaceutically compatible preparation” and “pharmaceutical composition”, as used herein, relate to compositions comprising the compounds of the present invention and optionally one or more pharmaceutically acceptable carrier. The compounds of the present invention can be formulated as pharmaceutically acceptable salts. Preferred acceptable salts are acetate, methylester, HCl, sulfate, chloride and the like. The pharmaceutical compositions are, preferably, administered topically or, more preferably, systemically. Suitable routes of administration conventionally used for drug administration are oral, intravenous, subcutaneous, or parenteral administration as well as inhalation. However, depending on the nature and mode of action of a compound, the pharmaceutical compositions may be administered by other routes as well. Moreover, the compounds can be administered in combination with other drugs either in a common pharmaceutical composition or as separated pharmaceutical compositions as specified elsewhere herein, wherein said separated pharmaceutical compositions may be provided in form of a kit of parts. Preferably, the combined preparation is an extended release preparation with regard to one of the compounds, wherein the term “extended release”, preferably, relates to a compound encapsulated in microspheres based, preferably, on Medisorb or similar microsphere technology (Kim M. R. et al., (2010), Chem. Comm. (Camb) 46: 7433).

The compounds are, preferably, administered in conventional dosage forms prepared by combining the drugs with standard pharmaceutical carriers according to conventional procedures. These procedures may involve mixing, granulating and compressing or dissolving the ingredients as appropriate for the desired preparation. It will be appreciated that the form and character of the pharmaceutically acceptable carrier or diluent is dictated by the amount of active ingredient with which it is to be combined, the route of administration and other well-known variables.

The carrier(s) must be acceptable in the sense of being compatible with the other ingredients of the formulation and being not deleterious to the recipient thereof. The pharmaceutical carrier employed may be, for example, a solid, a gel or a liquid. Exemplary of solid carriers are lactose, terra alba, sucrose, talc, gelatin, agar, pectin, acacia, magnesium stearate, stearic acid, degradable polymers like PLGA (DeYoung at al. (2011), DIABETES TECHNOLOGY & THERAPEUTICS 13:1145; Ramazani et al., (2016), Int J Pharm. 499(1-2): 358-367, and the like. Exemplary liquid carriers are phosphate buffered saline solution, syrup, oil such as peanut oil and olive oil, water, emulsions, various types of wetting agents, sterile solutions and the like. Similarly, the carrier or diluent may include time delay material well known to the art, such as glyceryl mono-stearate or glyceryl distearate alone or with a wax. Said suitable carriers comprise those mentioned above and others well known in the art, see, e.g., Remington's Pharmaceutical Sciences, Mack Publishing Company, Easton, Pa.

The diluent(s) is/are selected so as not to affect the biological activity of the compound or compounds. Examples of such diluents are distilled water, physiological saline, Ringer's solutions, dextrose solution, and Hank's solution. In addition, the pharmaceutical composition or formulation may also include other carriers, adjuvants, or nontoxic, nontherapeutic, nonimmunogenic stabilizers, reactive oxygen scavengers, and the like. In a preferred embodiment, the pharmaceutical composition comprises an agent inducing the cAMP pathway of a host cell, preferably a mammalian cell. Inducers of the cAMP pathway are known in the art and include in particular activators of adenylyl cyclase, preferably forskolin (CAS 66428-89-5), inhibitors of phosphodiesterase, e.g. caffeine, and cAMP analogues, preferably 8-(4-Chlorophenylthio)-adenosine-3′,5′-cyclic monophosphate (8-CPT-cAMP, CAS 93882-12-3) or 8-Bromoadenosine 3′,5′-cyclic monophosphate (CAS 23583-48-4). Preferably, the agent inducing the cAMP pathway is forskolin or 8-CPT-cAMP.

A therapeutically effective dose refers to an amount of the compounds to be used in a pharmaceutical composition of the present invention which prevents, ameliorates or treats the symptoms accompanying a disease or condition referred to in this specification. Therapeutic efficacy and toxicity of such compounds can be determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, LD₅₀/ED₅₀.

The dosage regimen will be determined by the attending physician and other clinical factors; preferably in accordance with any one of the above described methods. As is well known in the medical arts, dosages for any one patient depends upon many factors, including the patient's size, body surface area, age, the particular compound to be administered, sex, time and route of administration, general health, and other drugs being administered concurrently. Progress can be monitored by periodic assessment. A typical dose can be, for example, in the range of 1 to 1000 μg; however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Preferably, extended release preparations of each drug are injected from once per 1 week to once per 2 months or even at longer intervals. Progress can be monitored by periodic assessment. Preferred doses and concentrations of the compounds of the present invention are specified elsewhere herein.

By means of example, a final concentration of Rotenone in tumor tissue preferably is not less than 1.25 nM. Preferably, the Rotenone concentration in blood is less than 1 μM. More preferably, Rotenone is administered in an extended release formulation, in particular from extended release microspheres in a monthly or bimonthly dose of from 5 μg/kg to 250 μg/kg, more preferably of from 14 μg/kg to 125 μg/kg. In a further non-limiting example, Papaverine hydrochloride may be administered in a single oral dose of Papaverine of from 50 mg to 150 mg, preferably 80 mg. The same dose may also be administered as intravenous injection for 5 min. A further preferred dosage of Papaverine is 1 to 25 mg/kg, more preferably 2.2-20 mg/kg.

In a further non-limiting example, preferred concentrations of Salinomycin, Monensin and Nigericin in tumor tissue are more than 1.25 nM. Salinomycin preferably is administered for treatment in human subjects at doses of from 100 to 300 μg/kg, more preferably 200 μg/kg intravenously every 2nd day, most preferably in an extended release formulation. Preferred doses of Nigericin are 2-3 times lower compared to Salinomycin. A further preferred dosage of Nigericin, Monensin and Salinomycin is 14 μg-125 μg/kg using subcutaneous delivery of extended release microspheres.

The pharmaceutical compositions and formulations referred to herein are, preferably, administered at least once, e.g. in case of extended release formulations, in order to treat or ameliorate or prevent a disease or condition recited in this specification. However, the said pharmaceutical compositions may be administered more than one time, for example from one to four times daily up to a non-limited number of days. Also some compounds with a short clearance time may be applied as infusion in blood stream to provide effective dose in whole body during long treatment time.

Specific pharmaceutical compositions are prepared in a manner well known in the pharmaceutical art and comprise at least one active compound referred to herein above in admixture or otherwise associated with a pharmaceutically acceptable carrier or diluent. For making those specific pharmaceutical compositions, the active compound(s) will usually be mixed with a carrier or the diluent, or enclosed or encapsulated in a capsule, sachet, cachet, paper or other suitable containers or vehicles. The resulting formulations are to be adopted to the mode of administration, i.e. in the forms of tablets, capsules, suppositories, solutions, suspensions or the like. Dosage recommendations shall be indicated in the prescribers or users instructions in order to anticipate dose adjustments depending on the considered recipient.

The present invention also relates to a medicament comprising (i) an inhibitor of mitochondrial respiration, (ii) a proton ionophore, and (iii) at least one pharmaceutically acceptable carrier; and to said medicament for use in treatment of cancer.

The term “medicament” is understood by the skilled person. As will be understood, the definitions given herein above for the term “combined preparation”, preferably, apply to the term medicament of the present invention mutatis mutandis.

Moreover, the present invention relates to a kit comprising an inhibitor of mitochondrial respiration and a proton ionophore, preferably comprised in a housing.

The term “kit”, as used herein, refers to a collection of the aforementioned components. Preferably, said components are combined with additional components, preferably within an outer container. The outer container, also preferably, comprises instructions for carrying out a method of the present invention. Examples for such the components of the kit as well as methods for their use have been given in this specification. The kit, preferably, contains the aforementioned components in a ready-to-use formulation. Preferably, the kit additionally comprises instructions, e.g., a user's manual for applying the inhibitor of mitochondrial respiration and the proton ionophore with respect to the applications provided by the methods of the present invention. Details are to be found elsewhere in this specification. Additionally, such user's manual may provide instructions about correctly using the components of the kit. A user's manual may be provided in paper or electronic form, e.g., stored on CD or CD ROM. The present invention also relates to the use of said kit in any of the methods according to the present invention. Moreover, the kit may also be used in a cell viability assay using cancer cells obtained from patient.

Further, the present invention relates to a method of treating cancer in a subject comprising

a) administering an inhibitor of mitochondrial respiration to said subject,

b) administering a proton ionophore to said subject,

c) thereby treating cancer in said subject.

The method of treating of the present invention, preferably, is an in vivo method. Moreover, it may comprise steps in addition to those explicitly mentioned above. For example, further steps may relate, e.g., to diagnosing cancer for step a), or administering further treatments, e.g. surgery, radiotherapy, and/or administration of cancer therapeutic agents before, simultaneously to, or after administering one or both of steps a) and b). Moreover, one or more of said steps may be performed by automated equipment.

In a preferred embodiment, the subject according to the present invention is a subject suffering from cancer as specified herein above. In a further preferred embodiment, the subject is a subject not suffering from diabetes type II.

As used herein, the term “cancer therapeutic agent” relates to an agent used to treat cancer. As used herein, the term cancer therapeutic agent is not used for an inhibitor of mitochondrial respiration and not for a proton ionophore, although these two groups of compounds are suitable in cancer therapy. The term cancer therapeutic agent, preferably, relates to a chemical substance known to inhibit growth of cancer cells, to kill cancer cells, or to cause the body of a patient to inhibit the growth of or to kill cancer cells in the treatment of cancer by application of said chemical substance to a patient in need thereof. More preferably, the cancer therapeutic agent is a chemotherapeutic agent, an agent for targeted therapy, an agent for immunotherapy, or any combination thereof.

As used herein, the term “chemotherapy” relates to treatment of a subject with an antineoplastic agent. Preferably, chemotherapy is a treatment including alkylating agents (e.g. cyclophosphamide), platinum (e.g. carboplatin), anthracyclines (e.g. doxorubicin, epirubicin, idarubicin, or daunorubicin) and topoisomerase II inhibitors (e.g. etoposide, irinotecan, topotecan, camptothecin, or VP16), anaplastic lymphoma kinase (ALK)-inhibitors (e.g. Crizotinib or AP26130), aurora kinase inhibitors (e.g. N-[4-[4-(4-Methylpiperazin-1-yl)-6-[(5-methyl-1H-pyrazol-3-yl)amino]pyrimidin-2-yl]sulfanylphenyl]cyclopropanecarboxamide (VX-680)), antiangiogenic agents (e.g. Bevacizumab), or Iodine 131-1-(3-iodobenzyl)guanidine (therapeutic metaiodobenzylguanidine), HDAC8-Inhibitors, Lactate dehydrogenase inhibitors, alone or any suitable combination thereof. It is to be understood that, preferably, chemotherapy relates to a complete cycle of treatment, i.e. a series of several (e.g. four, six, or eight) doses of antineoplastic drug or drugs applied to a subject separated by several days or weeks without such application.

The term “targeted therapy”, as used herein, relates to application to a patient a chemical substance known to block growth of cancer cells by interfering with specific molecules known to be necessary for tumorigenesis or cancer or cancer cell growth. Examples known to the skilled artisan are small molecules like, e.g. Bcl-2-Inhibitors (e.g. Obatoclax) and PARP-inhibitors (e.g. Iniparib), or monoclonal antibodies like, e.g., Rituximab or Trastuzumab.

The term “immunotherapy” as used herein relates to the treatment of cancer by modulation of the immune response of a subject. Said modulation may be inducing, enhancing, or suppressing said immune response.

Furthermore, the present invention relates to a method for determining whether a subject suffering from cancer is susceptible to a combined treatment comprising administration of an inhibitor of mitochondrial respiration and a proton ionophore, comprising

a) detecting in a sample of cancer cells of said subject whether said cancer cells are sensitive to combined treatment with an inhibitor of mitochondrial respiration and a proton ionophore, preferably detecting in a sample of cancer cells of said subject (i) whether said cancer is a Wnt signaling-dependent cancer and/or (ii) whether said cancer cells show an unfolded protein response upon administration of an inhibitor of mitochondrial respiration and a proton ionophore and/or decreased Sox4 expression, and

b) based on the result of the detection of step a), determining whether said subject suffering from cancer is susceptible to a combined treatment comprising administration of an inhibitor of mitochondrial respiration and a proton ionophore.

The method for determining whether a subject is susceptible to a combined treatment according to the present invention, preferably, is an in vitro method. Moreover, it may comprises further steps in addition to those explicitly mentioned above. For example, further steps may relate, e.g., to obtaining cancer cells from a sample before step a), or providing a recommendation for cancer therapy for the subject examined. Thus, preferably, the method for determining whether a subject is susceptible to a combined treatment is a method for providing information useful in deciding in further therapy of a subject. As will be understood by the skilled person, the method for determining whether a subject is susceptible to a combined treatment, preferably, is a method for providing relevant information to the medical practitioner, however, more preferably, does not provide a diagnosis and/or decision on therapy.

Means and methods for determining whether cells, in particular cancer cells, are sensitive to combined treatment with an inhibitor of mitochondrial respiration and a proton ionophore have been described herein above and include, preferably, comparing viability measured as ATP level of said cells in the presence and absence of an effective concentration of said inhibitor of mitochondrial respiration and proton ionophore. Preferably, in case cells are determined to have a statistically significant decreased viability and/or ATP level, said cells are determined to be susceptible to the combined treatment as related to herein.

Means and methods for determining whether a cancer is a Wnt signaling-dependent cancer have been described herein above and in the Examples. Preferably, in case cells determined to contain a statistically significant increased amount of b-catenin in nuclei and/or cytoplasm as compared to a reference of a normal cell, said cells are determined to be Wnt-signaling dependent and, preferably, are determined to be susceptible to the combined treatment as related to herein. In preferred embodiment, said determination of increased b-catenin expression is performed as described by Gomez-Millan (2014), BMC Cancer. 14:192.

Means and methods for determining whether cells, in particular cancer cells, show an unfolded protein response or decrease in Sox4 expression upon administration of an inhibitor of mitochondrial respiration and a proton ionophore have been described herein above and in the Examples. Preferably, in case cells are determined comprise an increased amount of said marker of unfolded protein response upon administration of an inhibitor of mitochondrial respiration and a proton ionophore as compared to a control without administration of an inhibitor of mitochondrial respiration and a proton ionophore, said cells are determined to be susceptible to the combined treatment as related to herein.

In a further preferred embodiment, the present invention relates to a method for determining whether a combined treatment comprising administration of an inhibitor of mitochondrial respiration and a proton ionophore is effective in a subject suffering from cancer, comprising

a) detecting in a sample of cancer cells of said subject (i) activity of the Wnt pathway, (ii) activity of the TGFb pathway, and/or (iii) the presence of cancer stem cells, and

b) based on the result of the detection of step a), determining whether said treatment is effective.

The method for determining whether a combined treatment is effective, preferably, is an in vitro method. Moreover, it may comprises further steps in addition to those explicitly mentioned above. For example, further steps may relate, e.g., to obtaining a sample and/or cancer cells from a sample before step a), or taking further diagnosis measures before or after the steps described.

Methods for determining the activity of the Wnt pathway are known to the skilled person and are described elsewhere herein. Preferably, determining Wnt pathway activity comprises determining Axin2 expression. Methods for determining the activity of the TGFbeta pathway are also known to the skilled person and are described elsewhere herein. Preferably, determining TGFbeta pathway acidity comprises determining SKIL expression. Methods for determining presence of cancer stem cells are also known to the skilled person. Preferably, determining the presence of cancer stem cells comprises determining LGR5 expression. Preferably, the aforesaid determination of Wnt pathway activity, of TGFbeta activity, and/or the presence of cancer stem cells is determined by detecting a gene product, preferably an RNA of at least one of the aforesaid genes. Thus, the method for determining whether a combined treatment is effective, preferably, comprises determining Axin2 expression, preferably Axin2 RNA expression; comprises determining SKIL expression, preferably SKIL RNA expression; and/or comprises determining LGR5 expression, preferably LGR5 RNA expression. In a preferred embodiment, said determining RNA expression comprises performing quantitative PCR.

In a further preferred embodiment, the present invention relates to a kit for determining whether a combined treatment comprising administration of an inhibitor of mitochondrial respiration and a proton ionophore is effective in a subject suffering from cancer, comprising (i) an agent for detecting the activity of the Wnt pathway in a sample of cancer cells, (ii) an agent for detecting activity of the TGFb pathway in a cancer cell, and/or (iii) an agent for detecting the presence of cancer stem cells.

Preferably, the agent for detecting of the kit for determining whether a combined treatment is effective is a polynucleotide, more preferably an oligonucleotide specifically hybridizing to one of the genes described herein above. Preferably, the kit comprises further features of the kit described elsewhere herein.

Moreover, the present invention relates to metformin for use in treatment of a Wnt signaling-dependent cancer.

In view of the above, the following embodiments are particularly envisaged:

1. An inhibitor of mitochondrial respiration for use in treatment of cancer with a proton ionophore.

2. The inhibitor of mitochondrial respiration for use of embodiment 1, wherein said inhibitor of mitochondrial respiration is an inhibitor of mitochondrial ATP production.

3. The inhibitor of mitochondrial respiration for use of embodiment 1 or 2, wherein said inhibitor of mitochondrial respiration is an inhibitor of mitochondrial complex I.

4. The inhibitor of mitochondrial respiration for use of any one of embodiments 1 to 3, wherein said inhibitor of mitochondrial respiration is (i) Papaverine (CAS Number: 61-25-6), (ii) Rotenone (CAS Number: 83-79-4), (iii) Annonacin (CAS Number:111035-65-5), (iv) 1-methyl 4-phenyl 1,2,3,6 tetrahydropyridine (CAS Number 23007-85-4), (v) 3-nitropropionic acid (CAS Number: 504-88-1), (vi) Piericidin A (CAS Number 2738-64-9), (vii) Bullatacin A (CAS Number 123123-32-0), (viii) Rolliniastatin-1 ((2S)-4-[(2R,13R)-2,13-dihydroxy-13-[(5 S)-5-[(2S)-5-[(1 S)-1-hydroxyundecyl]oxolan-2-yl]oxolan-2-yl]tridecyl]-2-methyl-2H-furan-5-one), (ix) Phenoxan (CAS No. 134332-63-1), (x) Thiangazole (CAS No. 138667-71-7), (xi) Idebenone CAS No. 58186-27-9), (xii) Aureothin (CAS No 2825-00-5), (xiii) β-lapachone, (xiv) a derivative of any one of (i) to (xiii), (xv) a pharmaceutically acceptable salt of any one of (i) to (xiv), or (xvi) a prodrug of any of any one of (i) to (xiii).

5. The inhibitor of mitochondrial respiration for use of any one of embodiments 1 to 4, wherein said proton ionophore is (I) Nigericin, (II) Salinomycin, (III) Monensin, (IV) Metformin, (V) Phenformin, (VI) Buformin, (VII) ionomycin, (VIII) carbonylcyanide-p-trifluoromethoxyphenylhydrazone (FCCP), (IX) carbonyl cyanide m-chlorophenyl hydrazone (CCCP), (X) Alborixin, (XI) Desmethylalborixin (X-206), (XII) Grisorixin, (XIII) a derivative of any one of (I) to (XII), (XIV) a prodrug of any of any one of (I) to (XII), or (XV) a pharmaceutically acceptable salt of any one of (I) to (XIII).

6. The inhibitor of mitochondrial respiration for use of any one of embodiments 1 to 5, wherein said cancer is lung cancer, breast cancer, colorectal carcinoma, skin cancer or prostate cancer.

7. The inhibitor of mitochondrial respiration for use of any one of embodiments 1 to 6, wherein said cancer is a cancer sensitive to combined treatment with an inhibitor of mitochondrial respiration and a proton ionophore.

8. The inhibitor of mitochondrial respiration for use of any one of embodiments 1 to 7, wherein said cancer sensitive to combined treatment with an inhibitor of mitochondrial respiration and a proton ionophore is a cancer showing a decrease in viability by at least 25% upon treatment with effective concentrations of inhibitor of mitochondrial respiration and proton ionophore.

9. The inhibitor of mitochondrial respiration for use of any one of embodiments 1 to 8, wherein said treatment of cancer is metastasis prevention.

10. A proton ionophore for use in treatment of cancer with an inhibitor of mitochondrial respiration.

11. The proton ionophore for use of embodiment 10, wherein said proton ionophore is (I) Nigericin, (II) Salinomycin, (III) Monensin, (IV) Metformin, (V) Phenformin, (VI) Buformin, (VII) ionomycin, (VIII) carbonylcyanide-p-trifluoromethoxyphenylhydrazone (FCCP), (IX) carbonyl cyanide m-chlorophenyl hydrazone (CCCP), (X) Alborixin, (XI) Desmethylalborixin (X-206), (XII) Grisorixin, (XIII) a derivative of any one of (I) to (XII), (XIV) a prodrug of any of any one of (I) to (XII), or (XV) a pharmaceutically acceptable salt of any one of (I) to (XIII).

12. The proton ionophore for use of embodiment 10 or 11, wherein said inhibitor of mitochondrial respiration is an inhibitor of mitochondrial ATP production.

13. The proton ionophore for use of any one of embodiments 10 to 12, wherein said inhibitor of mitochondrial respiration is an inhibitor of mitochondrial complex I.

14. The proton ionophore for use of any one of embodiments 10 to 13, wherein said inhibitor of mitochondrial respiration is (i) Papaverine (CAS Number: 61-25-6), (ii) Rotenone (CAS Number: 83-79-4), (iii) Annonacin (CAS Number:111035-65-5), (iv) 1-methyl 4-phenyl 1,2,3,6 tetrahydropyridine (CAS Number 23007-85-4), (v) 3-nitropropionic acid (CAS Number: 504-88-1), (vi) Piericidin A (CAS Number 2738-64-9), (vii) Bullatacin A (CAS Number 123123-32-0), (viii) Rolliniastatin-1 ((2S)-4-[(2R,13R)-2,13-dihydroxy-13-[(5S)-5-[(2S)-5-[(1S)-1-hydroxyundecyl]oxolan-2-yl]oxolan-2-yl]tridecyl]-2-methyl-2H-furan-5-one), (ix) Phenoxan (CAS No. 134332-63-1), (x) Thiangazole (CAS No. 138667-71-7), (xi) Idebenone CAS No. 58186-27-9), (xii) Aureothin (CAS No 2825-00-5), (xiii) β-lapachone, (xiv) a derivative of any one of (i) to (xiii), (xv) a pharmaceutically acceptable salt of any one of (i) to (xiv), or (xvi) a prodrug of any of any one of (i) to (xiii).

15. The proton ionophore for use of any one of embodiments 10 to 14, wherein said cancer is lung cancer, breast cancer skin cancer, prostate cancer, or colorectal carcinoma.

16. The proton ionophore for use of any one of embodiments 10 to 15, wherein said cancer is a cancer sensitive to combined treatment with an inhibitor of mitochondrial respiration and a proton ionophore.

17. The proton ionophore for use of any one of embodiments 10 to 16, wherein said cancer sensitive to combined treatment with an inhibitor of mitochondrial respiration and a proton ionophore is a cancer showing a decrease in viability by at least 25% upon treatment with effective concentrations of inhibitor of mitochondrial respiration and proton ionophore.

18. The proton ionophore for use of any one of embodiments 10 to 17, wherein said treatment of cancer is metastasis prevention.

19. A combined preparation for simultaneous, separate or sequential use comprising (i) an inhibitor of mitochondrial respiration and (ii) a proton ionophore.

20. The combined preparation of embodiment 19, wherein said inhibitor of mitochondrial respiration is an inhibitor of mitochondrial ATP production.

21. The combined preparation of embodiment 19 or 20, wherein said inhibitor of mitochondrial respiration is an inhibitor of mitochondrial complex I.

22. The combined preparation of any one of embodiments 19 to 21, wherein said inhibitor of mitochondrial respiration is (i) Papaverine (CAS Number: 61-25-6), (ii) Rotenone (CAS Number: 83-79-4), (iii) Annonacin (CAS Number:111035-65-5), (iv) 1-methyl 4-phenyl 1,2,3,6 tetrahydropyridine (CAS Number 23007-85-4), (v) 3-nitropropionic acid (CAS Number: 504-88-1), (vi) Piericidin A (CAS Number 2738-64-9), (vii) Bullatacin A (CAS Number 123123-32-0), (viii) Rolliniastatin-1 ((2S)-4-[(2R,13R)-2,13-dihydroxy-13-[(5S)-5-[(2S)-5-[(1 S)-1-hydroxyundecyl]oxolan-2-yl]oxolan-2-yl]tridecyl]-2-methyl-2H-furan-5-one), (ix) Phenoxan (CAS No. 134332-63-1), (x) Thiangazole (CAS No. 138667-71-7), (xi) Idebenone CAS No. 58186-27-9), (xii) Aureothin (CAS No 2825-00-5), (xiii) β-lapachone, (xiv) a derivative of any one of (i) to (xiii), (xv) a pharmaceutically acceptable salt of any one of (i) to (xiv), or (xvi) a prodrug of any of any one of (i) to (xiii).

23. The combined preparation of any one of embodiments 19 to 22, wherein said proton ionophore is (I) Nigericin, (II) Salinomycin, (III) Monensin, (IV) Metformin, (V) Phenformin, (VI) Buformin, (VII) ionomycin, (VIII) carbonylcyanide-p-trifluoromethoxyphenylhydrazone (FCCP), (IX) carbonyl cyanide m-chlorophenyl hydrazone (CCCP), (X) Alborixin, (XI) Desmethylalborixin (X-206), (XII) Grisorixin, (XIII) a derivative of any one of (I) to (XII), (XIV) a prodrug of any of any one of (I) to (XII), or (XV) a pharmaceutically acceptable salt of any one of (I) to (XIII).

24. A combined preparation according to any one of embodiments 19 to 23 for use in treatment of cancer.

25. The combined preparation for use of embodiment 24, wherein said cancer is lung cancer, breast cancer, skin cancer, prostate cancer or colorectal carcinoma.

26. The combined preparation for use of embodiment 24 or 25, wherein said cancer is a cancer sensitive to combined treatment with an inhibitor of mitochondrial respiration and a proton ionophore.

27. The combined preparation for use of any one of embodiments 24 to 26, wherein said cancer sensitive to combined treatment with an inhibitor of mitochondrial respiration and a proton ionophore shows a decrease in viability by at least 25% upon treatment with effective concentrations of inhibitor of mitochondrial respiration and proton ionophore.

28. The combined preparation for use of embodiment 24 to 27, wherein said treatment of cancer is metastasis prevention.

29. The combined preparation for use of embodiment 24 to 28, wherein said combined preparation comprises Rotenone and nigericin or comprises salinomycin and papaverine.

30. A medicament comprising (i) an inhibitor of mitochondrial respiration, (ii) a proton ionophore, and (iii) at least one pharmaceutically acceptable carrier.

31. The medicament of embodiment 30, wherein said inhibitor of mitochondrial respiration is an inhibitor of mitochondrial ATP production.

32. The medicament of embodiment 30 or 31, wherein said inhibitor of mitochondrial respiration is an inhibitor of mitochondrial complex I.

33. The medicament of any one of embodiments 30 to 32, wherein said inhibitor of mitochondrial respiration is (i) Papaverine (CAS Number: 61-25-6), (ii) Rotenone (CAS Number: 83-79-4), (iii) Annonacin (CAS Number:111035-65-5), (iv) 1-methyl 4-phenyl 1,2,3,6 tetrahydropyridine (CAS Number 23007-85-4), (v) 3-nitropropionic acid (CAS Number: 504-88-1), (vi) Piericidin A (CAS Number 2738-64-9), (vii) Bullatacin A (CAS Number 123123-32-0), (viii) Rolliniastatin-1 ((2S)-4-[(2R,13R)-2,13-dihydroxy-13-[(5S)-5-[(2S)-5-[(1 S)-1-hydroxyundecyl]oxolan-2-yl]oxolan-2-yl]tridecyl]-2-methyl-2H-furan-5-one), (ix) Phenoxan (CAS No. 134332-63-1), (x) Thiangazole (CAS No. 138667-71-7), (xi) Idebenone CAS No. 58186-27-9), (xii) Aureothin (CAS No 2825-00-5), (xiii) β-lapachone, (xiv) a derivative of any one of (i) to (xiii), (xv) a pharmaceutically acceptable salt of any one of (i) to (xiv), or (xvi) a prodrug of any of any one of (i) to (xiii).

34. The medicament of any one of embodiments 30 to 33, wherein said proton ionophore is (I) Nigericin, (II) Salinomycin, (III) Monensin, (IV) Metformin, (V) Phenformin, (VI) Buformin, (VII) ionomycin, (VIII) carbonylcyanide-p-trifluoromethoxyphenylhydrazone (FCCP), (IX) carbonyl cyanide m-chlorophenyl hydrazone (CCCP), (X) Alborixin, (XI) Desmethylalborixin (X-206), (XII) Grisorixin, (XIII) a derivative of any one of (I) to (XII), (XIV) a prodrug of any of any one of (I) to (XII), or (XV) a pharmaceutically acceptable salt of any one of (I) to (XIII).

35. A medicament according to any one of embodiments 30 to 34 for use in treatment of cancer.

36. The medicament for use of embodiment 35, wherein said cancer is lung cancer, breast cancer, skin cancer, prostate cancer or colorectal carcinoma.

37. The medicament for use of embodiment 35 or 36, wherein said cancer is a cancer sensitive to combined treatment with an inhibitor of mitochondrial respiration and a proton ionophore.

38. The medicament for use of any one of embodiments 35 to 37, wherein said treatment of cancer is metastasis prevention.

39. A kit comprising an inhibitor of mitochondrial respiration and a proton ionophore, preferably comprised in a housing.

40. The kit of embodiment 39, wherein said inhibitor of mitochondrial respiration is an inhibitor of mitochondrial ATP production.

41. The kit of embodiment 39 or 40, wherein said inhibitor of mitochondrial respiration is an inhibitor of mitochondrial complex I.

42. The kit of any one of embodiments 39 to 41, wherein said inhibitor of mitochondrial respiration is (i) Papaverine, (ii) Rotenone, (iii) Annonacin, (iv) a derivative of any one of (i) to (iii), (v) a pharmaceutically acceptable salt of any one of (i) to (iii), or (vi) a prodrug of any of any one of (i) to (iii).

43. The kit of any one of embodiments 39 to 42, wherein said proton ionophore is (I) Nigericin, (II) Salinomycin, (III) Monensin, (IV) Metformin, (V) Phenformin, (VI) Buformin, (VII) ionomycin, (VIII) carbonylcyanide-p-trifluoromethoxyphenylhydrazone (FCCP), (IX) carbonyl cyanide m-chlorophenyl hydrazone (CCCP), (X) Alborixin, (XI) Desmethylalborixin (X-206), (XII) Grisorixin, (XIII) a derivative of any one of (I) to (XII), (XIV) a prodrug of any of any one of (I) to (XII), or (XV) a pharmaceutically acceptable salt of any one of (I) to (XIII).

44. The kit of any one of embodiments 39 to 43, further comprising instructions on administering said inhibitor of mitochondrial respiration therapy, instructions on administering proton ionophore therapy and/or instructions on administering a combined inhibitor of mitochondrial respiration and proton ionophore therapy.

45. A method of treating cancer in a subject comprising

a) administering an inhibitor of mitochondrial respiration to said subject,

b) administering a proton ionophore to said subject,

c) thereby treating cancer in said subject.

46. The method of embodiment 45, wherein said cancer is lung cancer, breast cancer, skin cancer, prostate cancer, or colorectal carcinoma.

47. The method of embodiment 45 or 46, wherein said cancer is a cancer sensitive to combined treatment with an inhibitor of mitochondrial respiration and a proton ionophore.

48. The method of any one of embodiments 45 to 47, wherein said treating cancer is preventing metastasis.

49. A method for determining whether a subject suffering from cancer is susceptible to a combined treatment comprising administration of an inhibitor of mitochondrial respiration and a proton ionophore, comprising

a) detecting in a sample of cancer cells of said subject whether said cancer cells are sensitive to combined treatment with an inhibitor of mitochondrial respiration and a proton ionophore, preferably detecting in a sample of cancer cells of said subject (i) whether said cancer is a Wnt signaling-dependent cancer and/or (iii) whether said cancer cells show an unfolded protein response upon administration of an inhibitor of mitochondrial respiration and a proton ionophore, and

b) based on the result of the detection of step a), determining whether said subject suffering from cancer is susceptible to a combined treatment comprising administration of an inhibitor of mitochondrial respiration and a proton ionophore.

50. The method of embodiment 49, wherein, in case said cancer is detected to be a cancer sensitive to combined treatment with an inhibitor of mitochondrial respiration and a proton ionophore in step a), it is determined that said subject is susceptible to a combined treatment comprising administration of an inhibitor of mitochondrial respiration and a proton ionophore.

51. Metformin for use in treatment of a Wnt signaling-dependent cancer.

52. The inhibitor of mitochondrial respiration for use of any one of embodiments 1 to 3, the proton ionophore for use of any one of embodiments 10 to 13, the combined preparation of any one of embodiments 19 to 21, the medicament of any one of embodiments 30 to 32, or the kit of any one of embodiments 39 to 41, wherein said inhibitor of mitochondrial respiration is selected from the list consisting of (i) Papaverine (CAS Number: 61-25-6), (ii) Rotenone (CAS Number: 83-79-4), (iii) Annonacin (CAS Number:111035-65-5), (iv) 1-methyl 4-phenyl 1,2,3,6 tetrahydropyridine (CAS Number 23007-85-4), (v) 3-nitropropionic acid (CAS Number: 504-88-1), (vi) Piericidin A (CAS Number 2738-64-9), (vii) Bullatacin A (CAS Number 123123-32-0), (viii) Rolliniastatin-1 ((2S)-4-[(2R,13R)-2,13-dihydroxy-13-[(5S)-5-[(2S)-5-[(1 S)-1-hydroxyundecyl]oxolan-2-yl]oxolan-2-yl]tridecyl]-2-methyl-2H-furan-5-one), (ix) Phenoxan (CAS No. 134332-63-1), (x) Thiangazole (CAS No. 138667-71-7), (xi) Idebenone (CAS No. 58186-27-9), (xii) Aureothin (CAS No 2825-00-5), (xiii) β-lapachone, (xiv) Phenformin (CAS Number:114-86-3), (xv) Metformin (CAS Number: 657-24-9), (xvi) Buformin (CAS Number:692-13-7), (xvii) NT1014, (xviii) Bay 87-2243 (CAS Number: 1227158-85-1), (xix) Gossypol (CAS Number: 303-45-7), (xx) a derivative of any one of (i) to (xix), (xxi) a pharmaceutically acceptable salt of any one of (i) to (xx), and (xxii) a prodrug of any one of (i) to (xix).

53. The inhibitor of mitochondrial respiration for use of any one of embodiments 1 to 3, the proton ionophore for use of any one of embodiments 10 to 13, the combined preparation of any one of embodiments 19 to 21, the medicament of any one of embodiments 30 to 32, or the kit of any one of embodiments 39 to 41, wherein said inhibitor of mitochondrial respiration is selected from the list consisting of (i) Papaverine, (ii) Rotenone, (iii) Annonacin, (iv) Phenformin, (v) Bay 87-2243, (vi) Gossypol, (vii) a derivative of any one of (i) to (vi), (viii) a pharmaceutically acceptable salt of any one of (i) to (vi), and (ix) a prodrug of any of any one of (i) to (vi).

54. The inhibitor of mitochondrial respiration for use of any one of embodiments 1 to 4, the proton ionophore for use of embodiment 10, the combined preparation of any one of embodiments 19 to 22, the medicament of any one of embodiments 30 to 33, or the kit of any one of embodiments 39 to 42, wherein said proton ionophore is selected from the list consisting of (I) Nigericin, (II) Salinomycin, (III) Monensin, (IV) Maduramicin, (V) Lasalocid, (VI) Narasin, (VII) ionomycin, (VIII) carbonylcyanide-p-trifluoromethoxyphenylhydrazone (FCCP), (IX) carbonyl cyanide m-chlorophenyl hydrazone (CCCP), (X) Alborixin (CAS: 57760-36-8), (XI) Desmethylalborixin (X-206, CAS 36505-48-3), (XII) Grisorixin (CAS: 31357-58-1), (XIII) Semduramicin, (XIV) a derivative of any one of (I) to (XIII), (XV) a prodrug of any of any one of (I) to (XIII), and (XVI) a pharmaceutically acceptable salt of any one of (I) to (XIV).

55. A method for determining whether a combined treatment comprising administration of an inhibitor of mitochondrial respiration and a proton ionophore is effective in a subject suffering from cancer, comprising

a) detecting in a sample of cancer cells of said subject (i) activity of the Wnt pathway, (ii) activity of the TGFb pathway, and/or (iii) the presence of cancer stem cells, and

b) based on the result of the detection of step a), determining whether said treatment is effective.

56. A kit for determining whether a combined treatment comprising administration of an inhibitor of mitochondrial respiration and a proton ionophore is effective in a subject suffering from cancer, comprising

(i) an agent for detecting the activity of the Wnt pathway in a sample of cancer cells, (ii) an agent for detecting activity of the TGFb pathway in a cancer cell, and/or (iii) an agent for detecting the presence of cancer stem cells.

All references cited in this specification are herewith incorporated by reference with respect to their entire disclosure content and the disclosure content specifically mentioned in this specification.

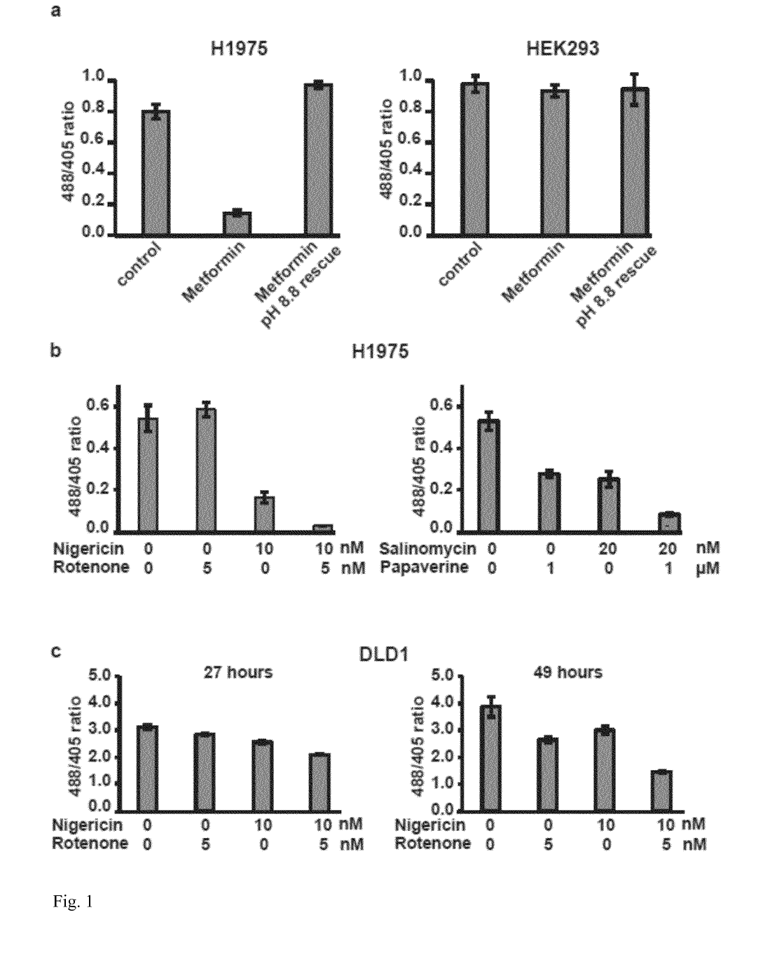

FIG. 1: Treatment with Metformin and combined preparations causes an intracellular pH drop in cancer cell lines. Intracellular pH measurements were done in cells expressing a pH sensitive variant of GFP (EC-GFP). Live cells were imaged at 405 nm and 488 nm excitation wavelengths to record control and pH-sensitive emissions, correspondingly. Eight biological replicates were done for each treatment. Error bars correspond to mean values±s.d.; P≤0.0001. a. Metformin causes intracellular acidification in cancer cells (H1975) but not in non-cancer cells (HEK293). Confocal microscopy of H1975 and HEK293 live cells expressing EC-GFP treated with Metformin for 48 hours. To rule out a possibility that GFP signal fading is due to degradation, after exposure to the drug, cells were transferred to media with pH 8.8 supplemented with 5 μM Nigericin for 30 minutes before re-imaging (Metformin pH 8.8 rescue). b, c. Cooperative effect of an ionophore drug and a mitochondrial complex I inhibitor on intracellular pH drop in H1975 (b) and DLD1 (c) cells. Intracellular pH measurements were done after cells were treated alone or in combination with Rotenone or/and Nigericin, and with Papaverine or/and Salinomycin for 27 or 48 hours in pH 6.5 buffer, as indicated.

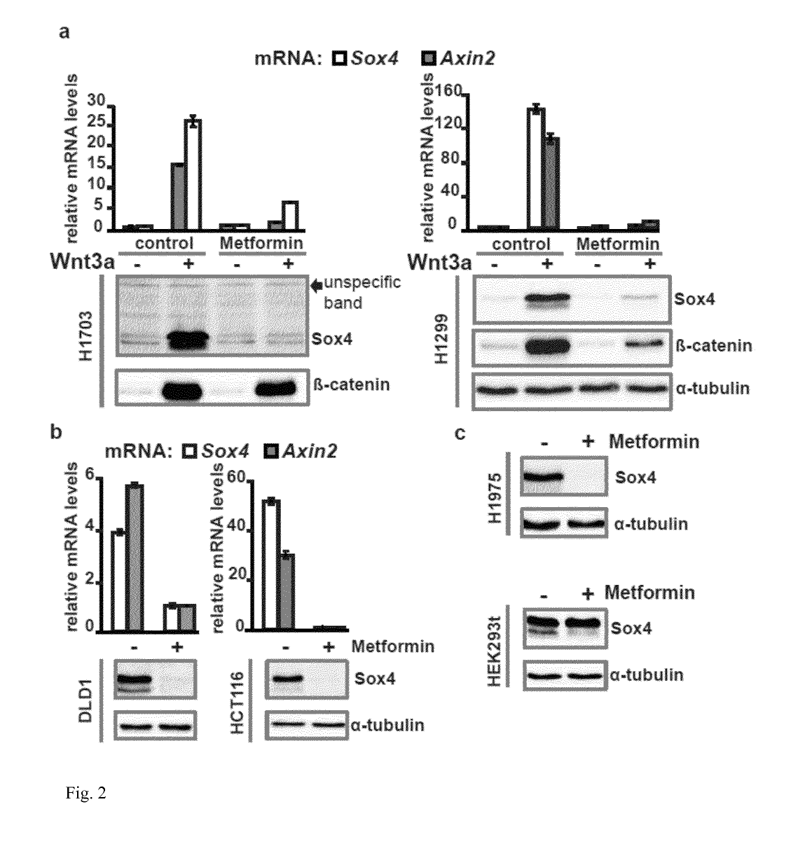

FIG. 2: Metformin inhibits expression of a general cancer marker gene Sox4 in cancer cell lines and has no effect in noncancer cells. Western blot and qRT-PCR analyses. a. Metformin inhibits Wnt-inducible Sox4. H1703 and H1299 cells were stimulated with Wnt3a conditioned media for 48 hours and treated with Metformin for 72 hours. Position of unspecific band used as a loading control for H1703 cells is indicated. b. Metformin inhibits expression of endogenous Sox4. DLD1 and HCT116 cells were treated with Metformin for 72 hours. c. Metformin inhibits expression of endogenous Sox4 in H1975 cells, and has no effect on Sox4 in non-malignant cell line, HEK293t. H1975 and HEK293t cells were treated with Metformin for 72 hours, as indicated.

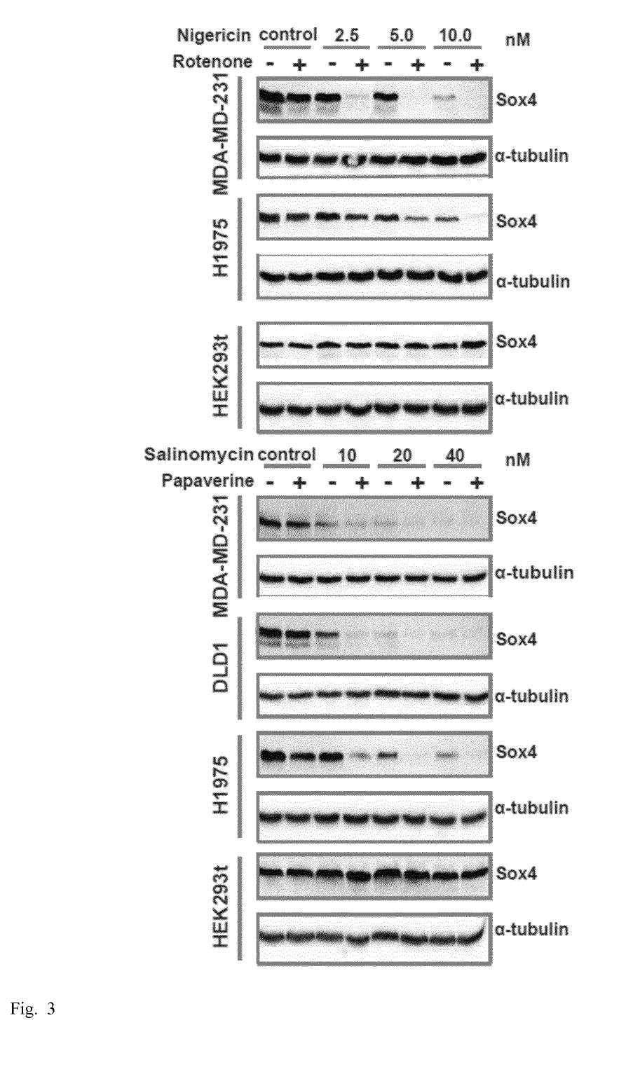

FIG. 3 The combined preparations inhibit protein expression of cancer marker gene Sox4 in cancer cell lines and have no effect in noncancer cells. a. Cooperation between ionophore drug and mitochondrial complex I inhibitor causes Sox4 inhibition. WB in cell lines treated with increasing concentrations of Nigericin in combination with 5 nM of Rotenone for 48 hours, as indicated. For all tested cancer cell lines, pH in culture media was 6.8 at the start of experiment, and, in case of non-cancer HEK293t cell line, pH was 7.4. Similarly, cells were treated with increasing concentrations of Salinomycin in combination with 0.5 μM of Papaverine for 48 hours.

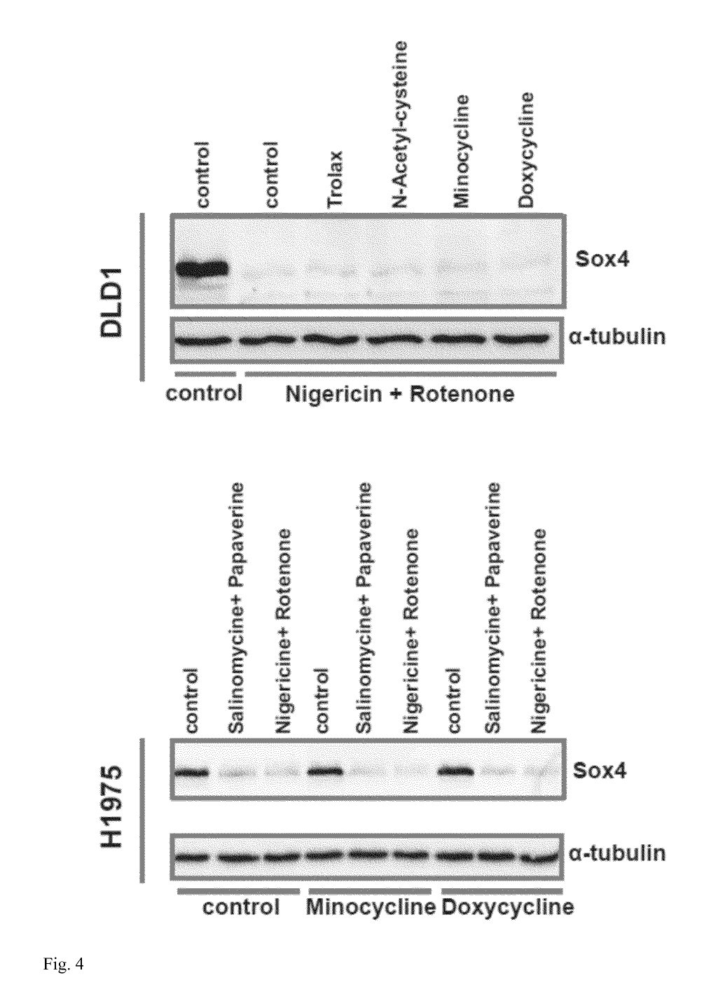

FIG. 4. Neuroprotectors (ROS scavengeres) do not interfere with Sox4 protein elimination by the combined preparations in cancer cell lines. a. Indicated cell lines were treated as in example 4 with invented drug combinations (Nigericin 10 nM, Rotenone 5 nM, Papaverine HCl 1 μm; Salinomycin 10 nM) with or without addition of NAC (N-acetyl cysteine) 1 mM, DOX (Doxycycline) and Minocycline 20 μM, Trolox 100 μM, as indicated.

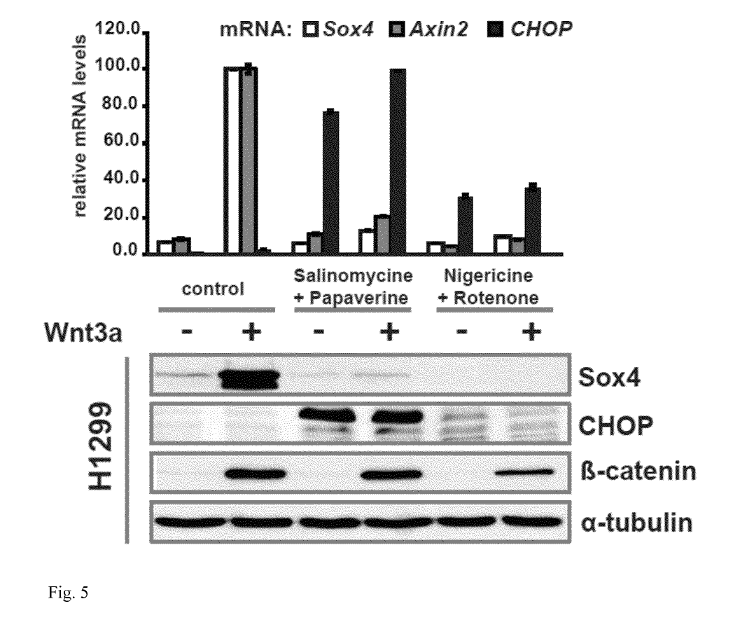

FIG. 5. Treatment with combined preparations inhibits Wnt signaling pathway. Western blot and qRT-PCR analyses in H1299 cells induced with Wnt3a conditioned medium for 48 hours in presence of drugs combination: 10 nM Salinomycin and 1 μM Papaverine, or 3 nM Nigericin and 5 nM Rotenone.

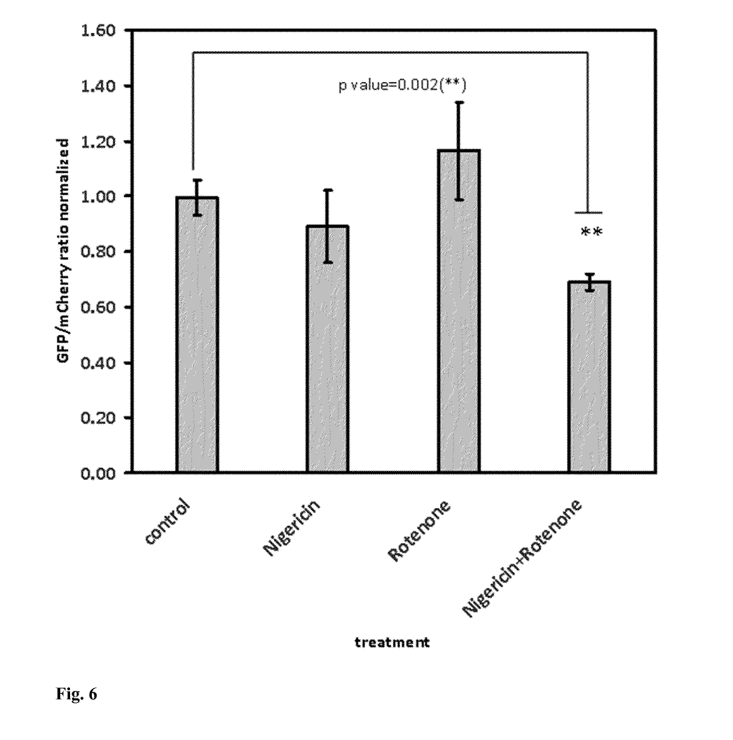

FIG. 6: Nigericin and Rotenone cooperatively drop pHᵢ in tumor xenografts (Example 7). Intracellular pH (y-axis) measurements were performed as described for FIG. 1, treatment as indicated, Nigericin was used at 5 μg/mg, Rotenone at 20 μg/mg.

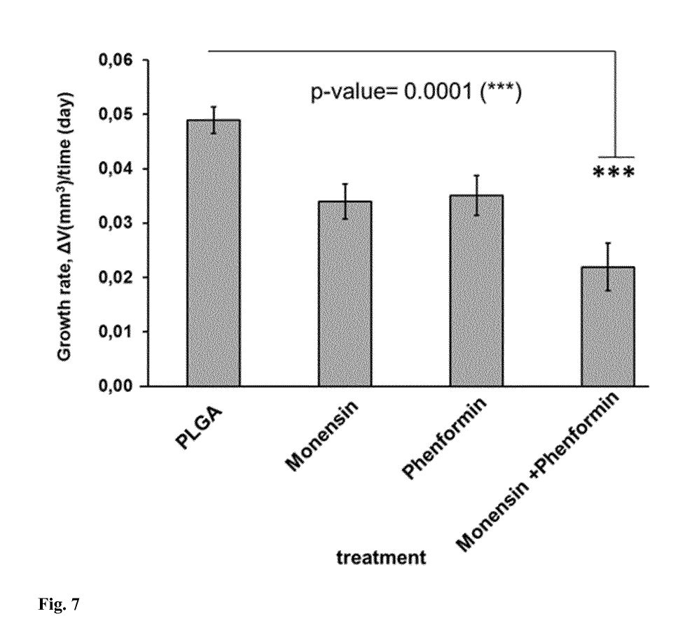

FIG. 7: Monensin and Phenformin cooperatively reduce growth rate of mouse tumor xenografts (Example 8). Tumor growth rate (y-axis) was measured for the xenografts of Example 8; Monensin was used at 1.14 mg/kg, Phenformin at 100 mg/kg.

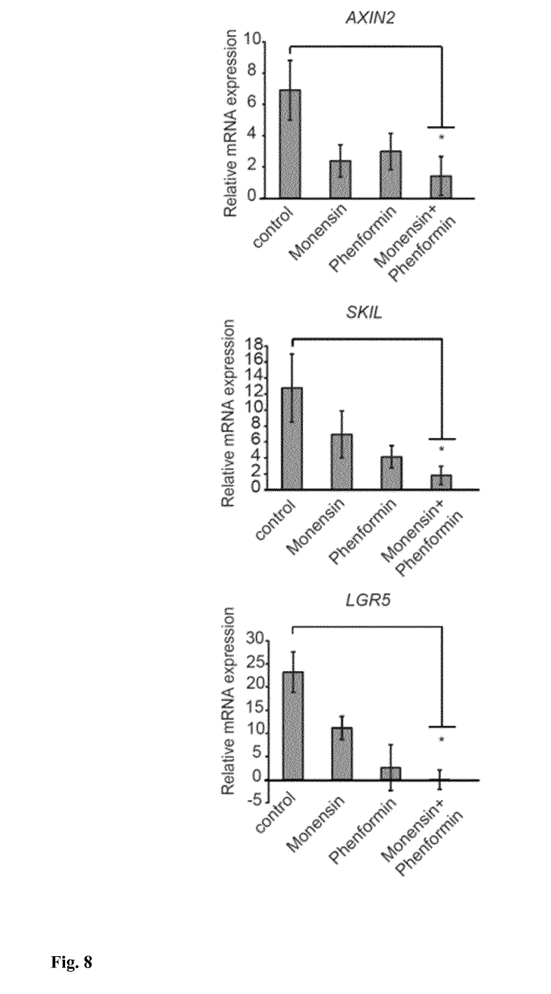

FIG. 8: Effect of treatments as described in Example 9 on expression of marker genes in tumor cells.

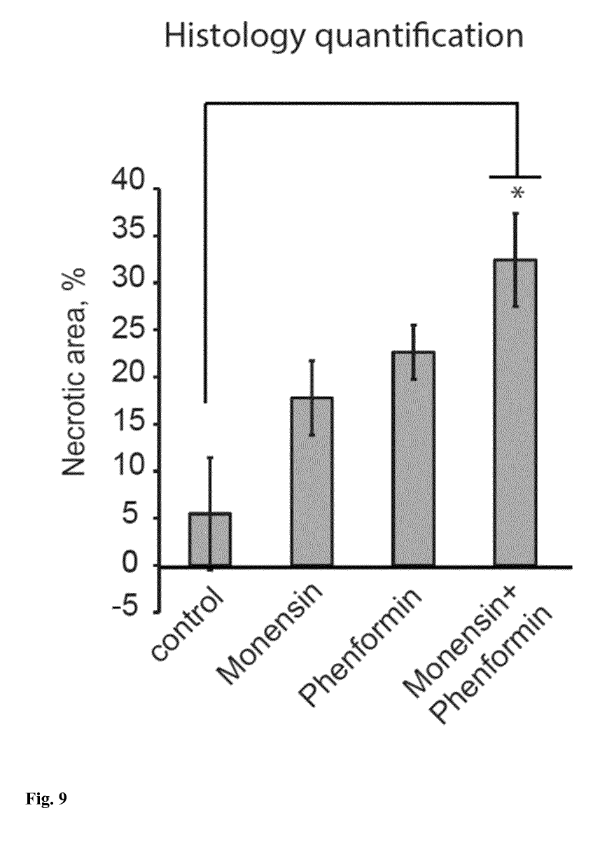

FIG. 9: Necrosis induction by the treatments as specified in Example 10.

The following Examples shall merely illustrate the invention. They shall not be construed, whatsoever, to limit the scope of the invention.

### EXAMPLE 1

Cancer cell viability was evaluated in cell culture experiments. Examples of Nigericin/Rotenone treatment using various cancer cell lines shows a synergistic (cooperative) effect of the selected drug pair Nigericin/Rotenone in killing cancer cells under conditions of tumor microenvironment (Tables 1-6) or Monensin/Rotenone (Table 7). Tables 1-5 show representative cell lines that are sensitive to invented treatments. For comparison, Table 6 shows an exceptional example of a cell line where treatment is effective, but a synergistic effect is not apparent. Table 8 shows that cAMP level increasing drugs potentiate invented combinations.

### Cell Viability Assay.

Indicated cells were treated with indicated drug combinations for 48 h or as indicated in conditions imitating tumor environment—RPMI supplemented with 10% FCS and 20 mM PIPES, adjusted to pH 6.5. After treatment, cells were analyzed for cell viability:
using CellTiter-Blue® Cell Viability Assay, Promega according to manufacturer recommendations. For each treatment, the fraction (%) of viable cells are indicated. Untreated viable cells were set to 100%. Average data of 2 independent experiments are presented. Each independent experiment contained 4 biological replicates for each condition.
using CellTiter-Glo® Cell Viability Assay, Promega according manufacturer recommendations. For each treatment the fraction (%) of viable cells are indicated. Untreated viable cells were set to 100%. Each measurement represents the mean value of 4 biological replicates for each condition.

TABLE 1
Synergistic killing of H1975 cells. CellTiter-Blue ® detection

| Lung | | Nigericin, nM | | |
|---|---|---|---|---|
| cancer H1975 cells | | 0.00 | 2.50 | 5.00 |
| Rotenone, nM | 0.00 | 100% | 93% | 95% |
| | 2.50 | 103% | 101% | 91% |
| | 5.00 | 104% | 97% | 102% |
| | 10.00 | 110% | 100% | 99% |
| | 20.00 | 84% | 54% | 59% |

TABLE 2
Synergistic killing of MDA-MB-231 cells. CellTiter-Blue ® detection.

| Breast cancer | | Nigericin, nM | | |
|---|---|---|---|---|
| MDA-MB-231 cells | | 0.00 | 2.50 | 5.00 |
| Rotenone, nM | 0.00 | 100% | 98% | 101% |
| | 2.50 | 98% | 97% | 99% |
| | 5.00 | 102% | 96% | 101% |
| | 10.00 | 99% | 93% | 97% |
| | 20.00 | 81% | 30% | 29% |

TABLE 3
Synergistic killing of colon cancer cells. CellTiter-Glo ® detection.

| Colon cancer | | | | |
|---|---|---|---|---|
| DLD1 cells, 72 h | | Nigericin, nM | | |
| treatment | | 0.00 | 5.00 | 10.00 |
| Rotenone, nM | 0.00 | 100% | 107% | 103% |
| | 5.00 | 96% | 66% | 59% |
| | 10.00 | 89% | 45% | 38% |
| | 20.00 | 67% | 30% | 22% |

TABLE 4
Synergistic killing of Melanoma cells. CellTiter-Glo ® detection.

| Melanoma | | Nigericin, nM | | |
|---|---|---|---|---|
| A375 cells | | 0.00 | 1.25 | 2.50 |
| Rotenone, nM | 0.00 | 100% | 58% | 7% |
| | 1.25 | 96% | 33% | 4% |
| | 2.50 | 91% | 10% | 2% |
| | 5.00 | 75% | 4% | 1% |

TABLE 5
Synergistic killing of Prostate cancer cells. CellTiter-Glo ® detection.

| Prostate cancer | | Nigericin, nM | | |
|---|---|---|---|---|
| PC3 cells | | 0.00 | 5.00 | 10.00 |
| Rotenone, nM | 0.00 | 100% | 31% | 0% |
| | 5.00 | 45% | 0% | 0% |
| | 10.00 | 4% | 0% | 0% |
| | 20.00 | 1% | 0% | 0% |

TABLE 6
No synergistic effect on Pancreas cancer cells. CellTiter-Glo ® detection.

| Pancreas cancer | | Nigericin, nM | | |
|---|---|---|---|---|
| Bxpc3 cells | | 0.00 | 5.00 | 10.00 |
| Rotenone, nM | 0.00 | 100% | 39% | 35% |
| | 5.00 | 104% | 47% | 49% |
| | 10.00 | 89% | 52% | 51% |
| | 20.00 | 59% | 51% | 54% |

TABLE 7
Synergistic killing of DLD1 cells. CellTiter-Blue ® detection

| Colon cancer DLD1 | | | Monensin, nM | | | |
|---|---|---|---|---|---|---|
| cells, 72 h treatment | | | 0.00 | 5.00 | 10.00 | 20.00 |
| | Rotenone, nM | 0.00 | 100% | 88% | 72% | 55% |
| | | 5.00 | 87% | 7% | 4% | 2% |

TABLE 8
Intracellular cAMP increasing drugs potentiate cancer cells killing effect
of invented combinations. Values are relative values obtained by
CellTiter-Glo ® detection of cell viability.

| Mouse melanoma | | | 8-CPT-cAMP |
|---|---|---|---|
| B16F10 | Control | Forskolin 10 μM | 100 μM |
| Control | 100% | 78% | 86% |
| Monensin 2.5 nM + | 88% | 19% | 10% |
| Rotenone 2.5 nM | | | |

TABLE 9
Synergistic killing of DLD1 cells. CellTiter-Glo ® detection

| Colon cancer DLD1 | | | Bay 87-2243 nM | | | |
|---|---|---|---|---|---|---|
| cells, 72 h treatment | | | 0.00 | 2.50 | 5.00 | 10.00 |
| | Monensin, nM | 0.00 | 100% | 96% | 90% | 26% |
| | | 5.00 | 112% | 35% | 6% | 4% |

TABLE 10
Synergistic killing of B16F10 cells. CellTiter-Glo ® detection

| Mouse melanoma | | | Metformin μM | | | |
|---|---|---|---|---|---|---|
| B16F10 | | | 0.00 | 750 | 1500 | 3000 |
| | Monensin, nM | 0.00 | 100% | 80% | 84% | 9% |
| | | 4.00 | 111% | 89% | 15% | 0% |

TABLE 11
Synergistic killing of B16F10 cells. CellTiter-Glo ® detection

| Mouse | | | Phenformin μM | | | |
|---|---|---|---|---|---|---|
| melanoma B16F10 | | | 0.00 | 4 | 8 | 16 |
| | Monensin, nM | 0.00 | 100% | 82% | 76% | 56% |
| | | 4.00 | 95% | 75% | 16% | 0% |

TABLE 12
Synergistic killing of B16F10 cells. CellTiter-Glo ® detection

| | | | Phenformin μM | | | |
|---|---|---|---|---|---|---|
| Mouse melanoma B16F10 | | | 0.00 | 4 | 8 | 16 |
| | Maduramicin, | 0.00 | 100% | 82% | 76% | 56% |
| | nM | 4.00 | 111% | 98% | 81% | 1% |

TABLE 10
Synergistic killing of H1975 cells. CellTiter-Glo ® detection

| Lung cancer H1975, | | | Metformin μM | | | |
|---|---|---|---|---|---|---|
| 72 h treatment | | | 0.00 | 94 | 188 | 375 |
| | Monensin, nM | 0.00 | 100% | 84% | 70% | 45% |
| | | 5.00 | 55% | 26% | 10% | 2% |

### EXAMPLE 2

Changes in intracellular pH were evaluated in various cancer cell lines and in non-cancer 293T cells. This example shows that the combined treatment of the present invention causes the intracellular pH to drop in cancer cells under conditions of tumor microenvironment. Surprisingly, it was found that Metformin alone is able to induce a pH to drop in cancer cells (H1975), but not in non-cancer HEK293 cells (FIG. 1a). Combinations of drugs cause a synergistic effect on intracellular pH (FIG. 1b,c).

### Intracellular pH Measurement Assay.

Intracellular pH in indicated cancer cell lines and non-cancer 293T cells was measured using cell lines stabile expressing pH sensitive EC-GFP at two different excitation wave length to obtain 488 nm/405 nm ratios. This ratio can be converted into an intracellular pH value using a standard curve (Miesenböck G et al. (1998), Nature 394(6689):192-5). For the experiments, cells were densely seeded on 96-well flat-bottom plates (BD 353376). Starting on the following day, cells were treated with indicated drug combinations for 48 h under conditions imitating tumor environment—RPMI supplemented with 10% FCS and 20 mM PIPES, adjusted to pH 6.5. For non-cancer 293Tcells, normal, non modified RPMI medium supplemented with 10% FCS was used. Live cells were imaged using LSM710 confocal microscope (Carl Zeiss) equipped with EC Plan-Neofluar DIC 10×/0.3 NA objective lens (Carl Zeiss). For each well, 4 fields were acquired. 405 nm and 488 nm lasers were used for excitation, and emission light was collected using 535/50 filter for each laser. 488/405 ratios were quantified using ImageJ (NIH) software.

### EXAMPLE 3

Sox4 is a transcription factor expressed in embryonic cells and it is also often expressed in cancer cells (Lin C. M. et al., (2013) PloS One 8: e67128). It also has been shown to be a cancer stem cell marker for breast cancer (Zhang J., et al. (2012) Cancer Res. 72: 4597). Additionally, the expression of Sox4 correlates with invasiveness and metastatic ability of cancer cells (Song G. D., et al. (2015) Tumor Biology 36: 4167).

Changes in expression of cancer marker Sox4 were evaluated upon treatment with Metformin in different cancer cell lines and non-cancer HEK293T cells using detection of Sox4 mRNA and protein. In addition, the mRNA level of wnt signaling marker Axin2 and control gene GAPDH were measured. For protein expression cytoplasmic level of wnt signaling marker b-catenin and control protein tubulin were measured. This example shows that Metformin treatment causes strong reduction in cancer marker Sox4. Also it shows that Metformin is a wnt signaling inhibitor (FIG. 2A) in cancer cell lines.

mRNA and Protein Measurement Assay

For gene expression measurement, cells were seeded in 6 well plates. On the following day, cells were treated with 6 mM Metformin for 48 h in normal RPMI medium supplemented with 10% FCS. Cells were harvested using Trypsin/EDTA and divided in up to 3 aliquots. A first aliquot was used for RNA preparation using RNeasy kit (Qiagen) and subsequent quantitative reverse transcription-PCR (qRT-PCR) analysis with LC480 LightCycler (Roche). A second aliquot was extracted with Saponin buffer (0.05% Saponin, 1 mM MgCl₂, 1×TBS, 2 mM ME, 1× protein inhibitor cocktail (Roche) for obtaining cytoplasmic protein fraction. This fraction was used for b-catenin protein detection. Third aliquot was extracted with Triton lysis buffer ((TBS (50 mM Tris pH7.4, 150 mM NaCl, 2.7 mM KCl), 1% Triton X-100, 2 mM β-mercaptoethanol (ME), 1 mM MgCl₂, 10 mM sodium pyrophosphate, 10 mM NaF, 1× proteinase inhibitor cocktail). This aliquot was used for Sox4 and a-tubulin (control) protein detection. For second and third aliquots 20-35 μl of buffer was used for 500 000 cells. After centrifugation extracts were used for Western blot analysis.

### EXAMPLE 4

Changes in expression of cancer marker Sox4 were evaluated upon treatment with the combined preparations in different cancer cell lines and non-cancer HEK293T cells using detection of Sox4 protein. This example shows that the combined preparations cause a drop in cancer marker Sox4 protein level in cancer cells under conditions of tumor microenvironment and have no effect in non-cancer cells. Combinations of drugs cause synergistic effect on Sox4 elimination.

Experiments were done as in example 3. Cells were densely seeded on 6-well plates. Starting on the following day, cells were treated with indicated drug combinations for 48 h in conditions imitating tumor environment: RPMI supplemented with 10% FCS and 20 mM PIPES, adjusted to pH 6.5. For non-cancer HEK293Tcells, non modified RPMI medium supplemented with 10% FCS was used. Cells were extracted only in Triton lysis buffer (as in example 3) and analyzed in Western Blot.

### EXAMPLE 5

Changes in expression of cancer marker Sox4 were evaluated upon treatment with combined preparations in various cancer cell lines in the presence of neuroprotection drugs—reactive oxygen species (ROS) scavengers. Effect on cancer marker Sox4 obtained by combined preparation treatment could not be rescued with well-known neuroprotection drugs. This suggests that neuroprotection drugs can be included in pharmaceutical formulations without reducing efficacy of treatment.

Experiments were done as in example 3. Cells were treated with combined preparations (Nigericin 10 nM, Rotenone 5 nM, Papaverine HCl 1 μm; Salinomycin 10 nM) with or without addition of NAC (N-acetyl cysteine) 1 mM, DOX (Doxycycline) and Minocycline 20 μM, Trolox (soluble form of vitamin E) 100 μM.

### EXAMPLE 6

The ability of combined preparations to inhibit wnt signaling was evaluated. This example shows that the combined preparations block wnt signaling induced in lung cancer cell line by application of condition medium containing Wnt3a protein. As read-out of wnt signaling induction, expression of wnt target genes, Sox4 and Axin2, was monitored. Simultaneously to Sox4 and Axin2 inhibition, CHOP gene is activated as a result of treatment with the combined preparations. Note that cytoplasmic b-catenin level is not affected by said treatments, confirming that signaling inhibition epistatically occurs downstream of b-catenin. This result provides support for use of the combined preparations in wnt-dependent cancers caused by APC or b-catenin mutations.

Experiment was done as in example 3. One day after plating, cells were treated with combined preparations combinations (Nigericin 3 nM, Rotenone 5 nM, Papaverine-HCl 1 μm; Salinomycin 10 nM) in Wnt3a conditioned medium or control medium, for 48 h. CHOP protein was detected in Triton lysis buffer extract.

### EXAMPLE 7

The ability of combined preparations to decrease the intracellular pH in vivo was evaluated. The example shows that the combined preparations are able, in cooperative manner, to decrease the intracellular pH in mouse tumor xenograft model. In vivo tumor imaging was performed with IVIS Lumina III (Perkin Elmer) system with 460 nm excitation and 520 nm emission filters to measure pH sensitive EC-GFP signal, and 580 nm excitation and 580 nm emission filterst to measure mCherry (normalization signal). Images were quantified with LivingImage software V4.4 (Caliper Life Sciences), and a ratios of EC-GFP to mCherry signals were calculated as a read-out of the intracellular pH changes (similar to example 2). This experiment provides an evidence that the combined preparations cause intracellular pH drop in vivo in the same manner as it was detected using cell culture.

Five to six-weeks old female NMRI nude mice (nu/nu) (purchased from Charles River, Sulzfeld, Germany) were subcutaneously injected with 2×10⁶ DLD1_EC-GFP/mCherry colon cancer cells resuspended in 100 μl PBS. After the tumor size reached 5 mm in either direction, mice were imaged, and the initial control GFP/mCherry ratios in engrafted tumors were measured. Animals were distributed into four randomized groups, with 5 mice per group, and were given, twice per week, intraperitoneal injections with 5 mg of PLGA microsphere preparations containing either Nigericin 5 μg/mg or Rotenone 20 μg/mg, or both drugs. A control treatment group was injected PLGA microsphere beads with no drug. One week later, animals were imaged again. After normalization to the control treatment and initial signals, EC-GFP/mCherry values were assessed using nonparametric t-test using GraphPad software. Error bars represent standard deviation values for 5 animals in group. ** p-value<0.01. Results are shown in FIG. 6.

### EXAMPLE 8

The ability of combined preparations to decrease tumor growth in vivo was evaluated. The example shows that the combined preparations are able, in cooperative manner, to decrease the rate of tumor growth in mouse tumor xenograft model. This experiment provides an evidence that the combined preparations significantly affect growth rate of tumor cells in vivo, similar to the effects demonstrated in example 1 using cell culture.

Five to six-weeks old female NSG albino mice (NOD scid gamma (NOD.Cg-Prkdcscid Il2rgtm1Wj1/SzJ), purchased from Charles River, Sulzfeld, Germany) were subcutaneously injected with 2×106 DLD1_EC-GFP/mCherry colon cancer cells resuspended in 100 μl PBS. After the tumor size reached 5 mm in either direction, mice were distributed into four randomized treatment groups, with 6 or 7 mice per group, and twice a week were given intraperitoneal injections with 5 mg/100 μl of either control PLGA microsphere preparations or PLGA preparations containing Monensin (20 μg/mg). Additionally, mice were either given drinking water or drinking water containing Phenformin (100 mg/kg). Tumor growth was monitored twice per week until it reached 15 mm in either direction, when mice were euthanized. Tumor growth rates were assessed using one-way ANOVA Dunnett's multiple comparison test. Error bars represent standard error values for 6 (PLGA) or 7 (all others groups) animals in a group. *** p-value<0.001. Results are shown in FIG. 7.

### EXAMPLE 9

The ability of combined preparations to inhibit pluripotency signaling pathways (Wnt and TGFβ) in vivo was evaluated. The example shows that the combined preparations are able, in cooperative manner, to inhibit mRNA expression of AXIN2, Wnt signaling target gene, SKIL, TGFβ signaling target gene and LGR5, stem cell marker gene, in tumor xenografts tissue from treated mice in experiment shown in Example 8. This experiment provides an evidence that the combined preparations significantly affect signaling pathways, associated with high pluripotency, including Wnt- and TGFβ, signaling pathways in vivo, similar to the effects demonstrated in Example 3 using cell culture. Additionally, a bona fide cancer stem cell marker gene, LRG5, is also affected.

Five to six-weeks old female NSG albino mice (NOD scid gamma (NOD.Cg-Prkdcscid Il12rgtm1Wj1/SzJ), purchased from Charles River, Sulzfeld, Germany) were subcutaneously injected with 2×106 DLD1_EC-GFP/mCherry colon cancer cells resuspended in 100 μl PBS. After the tumor size reached 5 mm in either direction, mice were distributed into four randomized treatment groups, with 6 (control) or 7 mice (all other groups) per group, and twice a week were given intraperitoneal injections with 5 mg/100 μl of either control PLGA microsphere preparations or PLGA preparations containing Monensin (20 μg/mg). Additionally, mice were either given drinking water or drinking water containing Phenformin (100 mg/kg). Tumor tissue was excised and snap-frozen in liquid nitrogen. Total RNA was isolated using an RNeasy Kit (Qiagen, Hilden, Germany) according to the manufacturer's instruction, with the DNaseI treatment step included. Relative mRNA expression was measured using quantitative RT-PCR with UPL probes (Roche) using LC480 LightCycler (Roche). mRNA values were normalized to the housekeeping gene GAPDH, and statistical significance of the data was evaluated using one-way ANOVA Dunnett's multiple comparison test. Error bars represent standard error values of median for 6 (control) or 7 (all others groups) animals in a group. * p-value<0.02. Results are shown in FIG. 8.

### EXAMPLE 10

The ability of combined preparations to affect tumor growth and induce necrosis in vivo was evaluated. The example shows that the combined preparations significantly induce necrosis of affected tumor xenograft tissues in treated mice in experiment shown in Example 8 and 9.

Five to six-weeks old female NSG albino mice (NOD scid gamma (NOD.Cg-Prkdcscid I12rgtm1Wj1/SzJ), purchased from Charles River, Sulzfeld, Germany) were subcutaneously injected with 2×10⁶ DLD1_EC-GFP/mCherry colon cancer cells resuspended in 100 μl PBS. After the tumor size reached 5 mm in either direction, mice were distributed into four randomized treatment groups, with 6 (control) or 7 mice (all other groups) per group, and twice a week were given intraperitoneal injections with 5 mg/100 μl of either control PLGA microsphere preparations (control) or PLGA preparations containing Monensin (20 μg/mg). Additionally, mice were either given drinking water or drinking water containing Phenformin (100 mg/kg). Tumor tissue was excised, and tumor necropsies were fixed in 4% paraformaldehyde/PBS for 16 hours and embedded into paraffin. 5 μm specimen were stained with Hematoxylin and Eosin according to a routine procedure. Images were acquired by a Zeiss Axioskop and processed with ImageJ Software to evaluate necrotic areas surfaces. For each tumor xenograft, 4 slices of specimen were prepared and evaluated. Statistical significance of the data was evaluated using one-way ANOVA Dunnett's multiple comparison test. Error bars represent standard error values of median for 6 (control) or 7 (all others groups) animals in a group. *p-value=0.025. Results are shown in FIG. 9.

1. A method of treating cancer in a subject comprising:
a) administering an inhibitor of mitochondrial respiration to said subject;
b) administering a proton ionophore to said subject; and
c) thereby treating cancer in said subject.

2. The method of claim 1, wherein said inhibitor of mitochondrial respiration is an inhibitor of mitochondrial ATP production.

3. The method of claim 1, wherein steps a) and b) are performed simultaneously.

4. The method of claim 1, wherein said inhibitor of mitochondrial respiration is selected from the list consisting of (i) Papaverine, (ii) Rotenone, (iii) Annonacin, (iv) 1-methyl 4-phenyl 1,2,3,6 tetrahydropyridine, (v) 3-nitropropionic acid, (vi) Piericidin A, (vii) Bullatacin A, (viii) Rolliniastatin-1, (ix) Phenoxan, (x) Thiangazole, (xi) Idebenone, (xii) Aureothin, (xiii) β-lapachone, (xiv) Phenformin, (xv) Metformin, (xvi) Buformin, (xvii) NT1014, (xviii) Bay 87-2243, (xix) Gossypol, (xx) a derivative of any one of (i) to (xix), (xxi) a pharmaceutically acceptable salt of any one of (i) to (xx), and (xxii) a prodrug of any of any one of (i) to (xix).

5. The method of claim 1, wherein said proton ionophore is selected from the list consisting of (I) Nigericin, (II) Salinomycin, (III) Monensin, (IV) Maduramicin, (V) Lasalocid, (VI) Narasin, (VII) ionomycin, (VIII) carbonylcyanide-p-trifluoromethoxyphenylhydrazone (FCCP), (IX) carbonyl cyanide m-chlorophenyl hydrazone (CCCP), (X) Alborixin, (XI) Desmethylalborixin, (XII) Grisorixin, (XIII) Semduramicin, (XIV) a derivative of any one of (I) to (XIII), (XV) a prodrug of any of any one of (I) to (XIII), and (XVI) a pharmaceutically acceptable salt of any one of (I) to (XIV).

6. The method of claim 1, wherein said cancer is lung cancer, breast cancer, or colorectal carcinoma.

7. The method of claim 1, wherein said cancer is a cancer sensitive to combined treatment with an inhibitor of mitochondrial respiration and a proton ionophore, preferably is a Wnt signaling-dependent cancer and/or is a cancer wherein the cells of said cancer show an unfolded protein response upon administration of an inhibitor of mitochondrial respiration and a proton ionophore.

8. (canceled)

9. A combined preparation for simultaneous, separate or sequential use comprising (i) an inhibitor of mitochondrial respiration and (ii) a proton ionophore.

10. The combined preparation of claim 9, wherein said inhibitor of mitochondrial respiration is selected from the list consisting of (i) Papaverine, (ii) Rotenone, (iii) Annonacin, (iv) 1-methyl 4-phenyl 1,2,3,6 tetrahydropyridine, (v) 3-nitropropionic acid, (vi) Piericidin A, (vii) Bullatacin A, (viii) Rolliniastatin-1, (ix) Phenoxan, (x) Thiangazole, (xi) Idebenone, (xii) Aureothin, (xiii) β-lapachone, (xiv) Phenformin, (xv) Metformin, (xvi) Buformin, (xvii) NT1014, (xviii) Bay 87-2243, (xix) Gossypol, (xx) a derivative of any one of (i) to (xix), (xxi) a pharmaceutically acceptable salt of any one of (i) to (xx), and (xxii) a prodrug of any of any one of (i) to (xix).

11. The combined preparation of claim 10, wherein said proton ionophore is selected from the list consisting of (I) Nigericin, (II) Salinomycin, (III) Monensin, (IV) Maduramicin, (V) Lasalocid, (VI) Narasin, (VII) ionomycin, (VIII) carbonylcyanide-p-trifluoromethoxyphenylhydrazone (FCCP), (IX) carbonyl cyanide m-chlorophenyl hydrazone (CCCP), (X) Alborixin, (XI) Desmethylalborixin, (XII) Grisorixin, (XIII) Semduramicin, (XIV) a derivative of any one of (I) to (XIII), (XV) a prodrug of any of any one of (I) to (XIII), and (XVI) a pharmaceutically acceptable salt of any one of (I) to (XIV).

12. The combined preparation of claim 10 for simultaneous administration.

13. (canceled)

14. A method for determining whether a subject suffering from cancer is susceptible to a combined treatment comprising administration of an inhibitor of mitochondrial respiration and a proton ionophore, comprising
a) detecting in a sample of cancer cells of said subject whether said cancer cells are sensitive to combined treatment with an inhibitor of mitochondrial respiration and a proton ionophore, preferably detecting in a sample of cancer cells of said subject (i) whether said cancer is a Wnt signaling-dependent cancer and/or (iii) whether said cancer cells show an unfolded protein response and/or said cancer cells show decrease in Sox4 expression upon administration of an inhibitor of mitochondrial respiration and a proton ionophore, and
b) based on the result of the detection of step a), determining whether said subject suffering from cancer is susceptible to a combined treatment comprising administration of an inhibitor of mitochondrial respiration and a proton ionophore.

15. (canceled)

16. The method of claim 1, further comprising administering surgery, radiotherapy, and/or administration of cancer therapeutic agents before, simultaneously to, or after administering one or both of steps a) and b).

17. The method of claim 1, wherein said cancer is a Wnt signaling-dependent cancer and wherein said method comprises administration of Metformin.
